(19) United States
(12) Patent Application Publication
(10) Pub. No.: US 2014/0322766 A1
(43) Pub. Date: Oct. 30, 2014

(54) C1-C2 Organic Acid Treatment of Lignocellulosic Biomass to Produce Acylated Cellulose Pulp, Hemicellulose, Lignin and Sugars and Fermentation of the Sugars

(71) Applicant: ARCHER DANIELS MIDLAND COMPANY, Decatur, IL (US)

(72) Inventors: Wuli Bao, Champaign, IL (US); Thomas Binder, Decatur, IL (US); Charles Abbas, Champaign, IL (US); Lucas Loveless, Chatham, IL (US)

(73) Assignee: ARCHER DANIELS MIDLAND COMPANY, Decatur, IL (US)

(21) Appl. No.: 14/279,559

(22) Filed: May 16, 2014

Related U.S. Application Data
(63) Continuation of application No. 14/342,634, filed on Mar. 4, 2014.
Application No. 14/342,634 is a continuation of application No. PCT/US12/56502, filed on Sep. 21, 2012.
(60) Provisional application No. 61/638,544, filed on Apr. 26, 2012.
(60) Provisional application No. 61/538,211, filed on Sep. 23, 2011.

(51) Int. Cl. C12P 7/14; D21C 3/04

(52) U.S. Cl. CPC C12P 7/14; D21C 3/04
USPC 435/115; 435/162; 435/136; 435/139; 435/137; 435/144; 435/145

(57) ABSTRACT

A process for production of C5 and C6 sugar enriched syrups from lignocellulosic biomass and fermentation products therefrom is described. A lignocellulosic biomass is treated with a C₁-C₂ acid (e.g., acetic acid) with washing thereof with a C₁-C₂ acid miscible organic solvent, (e.g., ethyl acetate). A soluble hemicellulose and lignin enriched fraction is obtained separately from a cellulose pulp enriched fraction and lignin is removed from the soluble hemicellulose fraction. These fractions contain acylated (e.g., acetylated) cellulose and hemicellulose, which are deacylated by treatment with an alkali and/or with an acetyl esterase enzyme. The deacylated fractions are then digested with suitable cellulolytic and/or hemicellulolytic enzymes, preferably in the presence of non-ionic detergent to yield the C5 and C6 enriched syrups. Also described are method of fermentation of the syrups to make ethanol to at least 7% w/vol by separate hydrolysis and fermentation (SHF) or simultaneous hydrolysis and fermentation (SSF) methods.

### CROSS REFERENCE TO RELATED APPLICATION[S]

This application is a continuation of pending U.S. application Ser. No. 14/342,634 filed on Mar. 4, 2014, which itself is a national phase entry of PCT application No. PCT/US12/56593 filed Sep. 21, 2012, which claims priority to U.S. provisional application No. 61/638,544 filed Apr. 26, 2012, which is incorporated herein by reference in its entirety. Pending U.S. application Ser. No. 14/342,634 also claims priority to PCT Application No. PCT/US12/56502 filed Sep. 21, 2012, which claims priority to provisional application No. 61/538,211 entitled Cellulolytic Enzyme Compositions and Uses Thereof filed Sep. 23, 2011, which also is incorporated herein by reference in its entirety.

### STATEMENT OF FEDERAL SPONSORED RESEARCH

This invention was made with government support under department of Energy Grant No: DE-EE0002870. The federal government has certain rights in this invention.

### BACKGROUND OF THE INVENTION

The hydrolysis of cellulose and hemicelluloses to monomeric sugars is a key perquisite to the commercial conversion of lignocellulosic feedstocks such as corn stover, corn fiber hulls, soybean hulls, wheat straws, sugarcane bagasse, sweet sugar beet pulp, and other forms of plant biomass derived from energy crops consisting of perennial grasses such as switch grass or miscanthus, bamboo and soft and/or hardwoods as well as pulp and waste paper residues. The conversion of lignocellulosic biomass to monomeric sugars, however, poses many technical challenges for economical uses of the monomeric sugars, especially as feedstocks for making products, such as ethanol, by fermentation of the sugars.

One of the technical problems is the process of hydrolysis itself. Conventionally, harsh acidic conditions using mineral acids coupled with high heat are required to hydrolyze a sufficient amount of the cellulose and hemicellulose polymers into their monomeric sugar residues to be useful. Unfortunately, such harsh conditions result in the production of numerous byproducts, such as fufural, hyrodxymethyl fufural, complex esters, humins, and tars that are toxic to microorganisms used for fermentation. Moreover, these by-products reduce the yield of monomeric sugars that can otherwise be obtained. These by-products could be separated from the monomeric sugars released by various purification techniques, but these add cost and result in still lower yields of useful sugars. Various cocktails of enzymes with cellulolytic and hemicellulolytic activity may be used in lieu of acid treatment or in conjunction with acid treatment, however the various available enzyme cocktails still do not provide cost-effective hydrolytic activity for a large fraction of the cellulose and hemicellulose present in typical lignocellulosic biomasses. Yet another technical problem is monomeric sugar preparations from lignocellulosic biomass contain relatively large amounts of C5 sugars such as xylose and a lesser quantity of arabinose derived from hemicellulose, as well as the C6 sugars mannose and galactose. Organisms that may otherwise be able to use such sugars for cell growth typically are inefficient in converting these sugars to the desired fermentation product in sufficient quantities to be economical. Even strains of yeast genetically engineered to utilize xylose are not able to accumulate ethanol in a typical fermentation process to more than 5% v/v, which is too low of yield to make the distillation of ethanol from the fermentation broth an economical proposition.

There is therefore still a need in the art to develop an integrated approach for preparing a lignocellulosic biomass of high quality that can be converted in high yield to C5 and C6 sugar feedstocks with reduced toxic byproducts, as well as methods of utilizing the rendered C5 and C6 sugar streams in fermentation protocols to achieve a combined ethanol yield of at least 8% v/v.

### SUMMARY OF THE INVENTION

The methods and materials made thereby as described herein overcome many of the foregoing technical challenges. The methods include use of a mild C₁-C₂ organic acid in conjunction with a suitable C₁-C₂ acid miscible organic solvent in initial rounds of hydrolysis to separate acid soluble hemicellulose and lignin from a cellulose pulp. The use of the C₁-C₂ organic acid results in esterification of the hemicellulose and cellulose, which is overcome by enzymatic and/or chemical de-esterification prior to, or in conjunction with, further hydrolysis of these fractions with an appropriate mixture of cellulolytic and hemicellulolytic enzymes. An esterase enzyme is included in preferred embodiments. The use of a non-ionic detergent in the enzymatic hydrolysis substantially increases the rate of catalytic conversion to suitable C5 and C6 enriched sugar syrups. Further these are used in staged fermentation processes to achieve greater than 8% ethanol production in the fermentation broth.

The results obtained were surprising in that contrary to published understanding from the prior art, the hydrolysis of cellulose to glucose can proceed without noticeable inhibition of cellulase enzyme activity and that ethanol concentrations over 5% are not detrimental to enzyme activities in the materials made by the methods described herein. In addition, the methods provide a way to make a useful cellulose pulp from monocot plant species, particularly wheat and corn stover, which have not been contemplated as source of cellulose pulp useful to replace pulp derived from more woody species.

A general embodiment is method of processing lignocellulosic biomass to form an acylated cellulose pulp that includes contacting a lignocellulosic biomass with a first amount of a C₁-C₂ acid selected from the group consisting of acetic acid, formic acid and mixtures of the same. The contacted lignocellulosic biomass is heated to a temperature and for a time sufficient to hydrolytically release a first portion of hemicellulose and lignin, forming a hydrolysate liquid and an acylated lignocellulose cake. The acylated lignocellulosic cake is separated from the first hydrolysate liquid and is contacted with a second amount of the C₁-C₂ acid to wash hemicellulose and lignin from the acylated lignocellulosic cake. The acid wash liquid including soluble hemicellulose and lignin is separated from the acid washed cake and the cake is contacted with a first amount of a C₁-C₂ acid-miscible organic solvent to further wash the C₁-C₂ acid, hemicellulose and lignin from the acid washed acylated cake leaving an acylated cellulose pulp, which is separated from the C₁-C₂ acid-miscible solvent wash liquid.

In a further embodiment the methods further include combining the solvent wash liquid with at least one of the hydrolysate and the acid wash liquid forming an acidic organic solvent extract. The acidic organic solvent extract is condensed forming an acidic organic solvent syrup enriched with hemicellulose and lignin. To that syrup, a second amount of the C1-C2 acid-miscible organic solvent is, the second amount being sufficient to form a precipitate comprised of hemicellulose and lignin. The precipitate of hemicellulose and lignin is separated from the acidic organic solvent syrup. The precipitate is mixed with an aqueous solvent to form a solution of solubilized hemicellulose and insoluble lignin and the insoluble lignin is separated from the solubilized hemicellulose.

In optional embodiments the hydrolysate is mixed with the acid wash liquid forming a mixture of acid soluble material prior to the condensing. In certain embodiment the condensing is by evaporation forming a vapor mixture comprising the C1-C2 acid and the C1-C2 acid miscible organic solvent. In a preferred practice of such embodiments the vapor mixture is recovered and the C1-C2 acid and the C1-C2 acid miscible organic are separated and recovered by distillation.

In certain embodiments after separating the hemicellulose and lignin precipitate from the acidic organic solvent syrup, a new amount of C1-C2 acid-miscible organic solvent is added to the acidic organic solvent syrup and a second hemicellulose and lignin containing precipitate is formed and also separated from acidic organic solvent syrup.

In further embodiments any of the above methods may further include contacting at least one of the solvent washed acylated cellulose pulp and the solubilized hemicellulose with a base forming at least one of a deacylated cellulose pulp and a deacylated hemicellulose fraction. In an preferred alternative, the deacylation includes contacting at least one of the solvent washed acylated cellulose pulp and the solubilized hemicellulose fraction with an esterase enzyme forming at least one of the deacylated cellulose pulp and a deacylated hemicellulose fraction.

Sill further embodiments include contacting at least one of the deacylated cellulose pulp and the deacylated hemicellulose fraction with an enzyme cocktail containing at least a hemicellulase and a cellulase enzyme for a time sufficient to form at least one syrup that is enriched in C5 or C6 sugars. In certain embodiments, contacting the solvent washed acylated cellulose pulp with an esterase enzyme occurs simultaneously with contacting with the enzyme cocktail containing at least a hemicellulase and a cellulase enzyme and wherein the esterase enzyme activity is supplemental to endogenous esterase enzyme activity present in the hemicellulase and cellulase enzyme cocktail. In particularly advantageous embodiments, contacting the washed acylated cellulose pulp in the presence of the esterase, hemicellulase or cellulase enzyme includes contacting in the presence of between 0.05% and 5% v/wt of a non-ionic detergent measured as a percentage of total weight of material present.

In preferred practices of the above methods, the lignocellulosic biomass is from a monocot species. In particular practices, the monocot species is selected from the group consisting of at least one of grasses, bamboo, wheat straw, corn stover, barley straw, millet straw, sorghum straw, and rice straw. In still more particular practices the monocot species is wheat straw, corn stover or bamboo.

In the exemplary embodiments, the C1-C2 acid is predominantly acetic acid, the acylated cellulose pulp comprises acetylated cellulose and the acylated hemicellulose comprises acetylated hemicellulose. Also in exemplary embodiments, the C1-C2 acid miscible organic solvent is predominantly ethyl acetate.

In one distinctive embodiment, the C1-C2 acid miscible organic solvent is not a halogenated organic solvent.

In most desirable practices the lignocellulosic biomass has a water content not greater than at least one of 40%, 20% or 10% wt/wt.

In another aspect there is provided an acylated or deacylated cellulose pulp or an acylated or deacylated hemicellulose made from a monocot species. In particular aspects of this embodiment the cellulose pulp is derived from a monocot species. In still more particular aspects the monocot species is selected from the group consisting of at least one of grasses, bamboo, wheat straw, corn stover, barley straw, millet straw, sorghum straw, and rice straw. In exemplary aspects, the cellulose pulp is derived from wheat straw, corn stover or bamboo. A cellulose pulp derived from wheat straw or corn stover provides a particularly new source of cellulose pulp that comes from an abundant low value by-product of grain production that is useful as a replacement or supplement to cellulose pulp derived from the more woody species of trees.

In yet another aspect there is provided a method of fermentation to make a desired fermentation product that includes obtaining at least one of an acylated cellulose pulp and acylated hemicellulose fraction, deacylating the acylated cellulose pulp and/or the acylated hemicellulose fraction, contacting the deacylated acylated cellulose pulp and/or acylated hemicellulose fraction with an enzyme cocktail comprising a mixture of at least two enzymes selected from the group consisting of a cellulase and a hemicellulase enzyme for a time sufficient to form a syrup comprised predominantly of C5 or C6 sugars and growing a microorganism on the sugars to produce the desired fermentation product.

In an integrated practice, obtaining the acylated cellulose pulp and/or acylated hemicellulose fraction is done according to the methods described herein above. In certain practices these include wherein deacylating the acylated cellulose pulp comprises contacting the obtained acylated cellulose pulp with a base. In certain preferred practices these include wherein deacylating the acylated cellulose pulp comprises contacting the obtained acylated cellulose pulp with an esterase. In some particularly preferred practices these include wherein contacting the acylated cellulose pulp with an esterase enzyme includes simultaneously contacting with the enzyme cocktail containing at least a hemicellulase and a cellulase enzyme wherein the esterase is supplemental to endogenous esterase contained in the enzyme cocktail. In certain preferred practices these include wherein contacting the acylated cellulose pulp or acylated hemicellulose in the presence of the esterase, hemicellulase or cellulase enzyme includes contacting in the presence of between 0.05% and 5% v/wt of a non-ionic detergent measured as a percentage of total weight of material present.

The fermentation methods include embodiments wherein growing the microorganism is done under conditions optimal to propagate the microorganism. In other embodiments, growing the microorganism is done under conditions optimal to produce the desired product by the microorganism.

In preferred fermentation method the desired product is ethanol and the microorganism is selected from the group consisting of Zymomonas mobilis and a yeast. The yeast is preferably S. cerevisiae.

When the organisms is a yeast and the growing may be done under aerobic conditions selected to propagate the yeast, particularly when the sugars are predominantly C5 sugars. In other practices where the organism is a yeast genetically engineered to ferment C5 sugars to form ethanol the growing is done under anaerobic conditions selected to produce ethanol.

While the fermentation methods are exemplified with ethanol as the desired product, the methods are equally applicable to other fermentation products. These include where the fermentation products are selected from the group consisting of an amino acid and an organic acid. When the fermentation product is an amino acid a typical amino acid is selected from the group consisting of lysine and threonine and the microorganism is selected from the group consisting of Escherichia coli and Corynebacterium glutanicum. When the preferred fermentation products are organic acids the organic acid may be selected from the group consisting of lactic acid, gluconoic acid, citric acid, malic acid, fumaric acid and succinic acid, in which case the microorganism is a fungus selected from the group consisting of Rhizopus, Schizosaccharomyces, Mucor, and Aspergillus.

### BRIEF DESCRIPTION OF THE DRAWINGS

FIG. 1 is a schemata for an overall embodiment of a biorefinery for processing lignocellulosic biomass to form a cellulose pulp, a hemicellulose fraction and a lignin fraction and subsequent formation of C5 and C6 sugars for use in making ethanol or other products by fermentation.

FIG. 2 illustrates an embodiment of a method incorporating a C₁-C₂ acid and C₁-C₂ acid-miscible organic solvent for preparation of a cellulose pulp, a hemicellulose fraction and a lignin fraction from lignocellulosic biomass.

FIG. 3 illustrates the difference in FTIR spectra of corn stover cellulose pulp (top trace) and ammonium hydroxide treated corn stover pulp (lower trace).

FIG. 4 shows the FTIR spectra between 1150 cm⁻¹ and 2000 cm⁻¹, where three important ester bonds are represented by the C═O ester stretching at 1725 cm⁻¹, the C—H stretching in —O(C═O)—CH₃ group at 1366 cm⁻¹, and the —CO— stretching of acetyl group at 1242 cm⁻¹ are indicated. The absence of a peak at 1700 cm⁻¹ representing the absorption of a carboxylic group confirmed that the alkaline treated sample is free of esterified acetic acid.

FIG. 5 illustrates the glucose being released by the enzyme reaction. After 7 days of incubation at 50° C., most of the glucose estimated to be present in the cellulose pulp 218 was released.

FIG. 6 illustrates a graphical summary of the data, including pulp dry solids, sugar concentration and ethanol concentration produced, from Tables 10-14 of the specification.

FIG. 7 illustrates one optimal method for a two stage semi-SSF process.

FIG. 8 is a graph illustrating the time course for production of ethanol and simultaneous utilization of the C5 sugar xylose during an exemplary first stage conducted in laboratory shake flasks in duplicate.

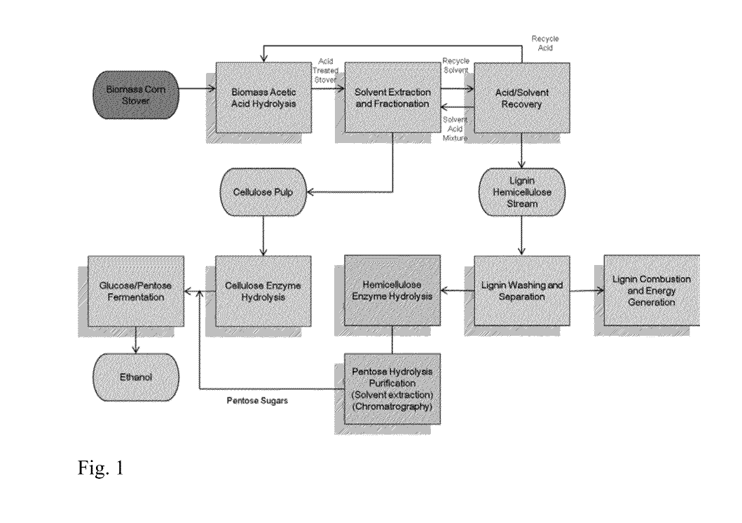

### DETAILED DESCRIPTION OF THE INVENTION

“Lignocellulosic biomass” means a plant material wherein the majority of the carbohydrates are in the form of cellulose and hemicellulose as distinct from starch and sugars. For the invention to be most workable the lignocellulosic biomass should have a moisture content of less than 40% and in typical embodiments the moisture content should be less than 30%, preferably less than 20% and most preferably less than 10%. Also it is preferable to use biomasses that have relatively low protein content because higher amounts of protein interfere with processing steps and contaminate the finally recovered hemicellulose and lignin fractions. The protein content should be less than 10% wt/wt of the biomass. Less than 5% is preferred in most embodiments. Suitable examples include wood, grasses, bamboo, the stalks of cereal grains such as wheat (straw), corn (stover), barley, millet, sorghum, and rice, as wells as the residual plant waste from harvesting dicotyledonous crops including some hulls of legumes and grains. Non suitable lignocellulosic biomass having too much protein include for example, corn hulls (a.k.a the “corn fiber” stream from a wet mill corn processing operation).

A “C₁-C₂ acid” means formic acid, acetic acid or mixtures of the same, which may include up to 30% water.

A “C₁-C₂ acid-miscible organic solvent” is a non-acidic organic solvent that is miscible with a C₁-C₂ acid and able to form a precipitate of hemicellulose and lignin from a C₁-C₂ acidic solution containing the same, with the proviso only that the C₁-C₂ acid-miscible organic solvent is not a halogenated solvent. Suitable examples include low molecular weight alcohols, ketones and esters, such as C₁-C₃ alcohols, acetone, ethyl acetate, methyl acetate, and methyl ethyl ketone.

“Acylate” and “acylated” means formation of an ester bond between a sugar or sugar residue of a polysaccharide and an organic acid.

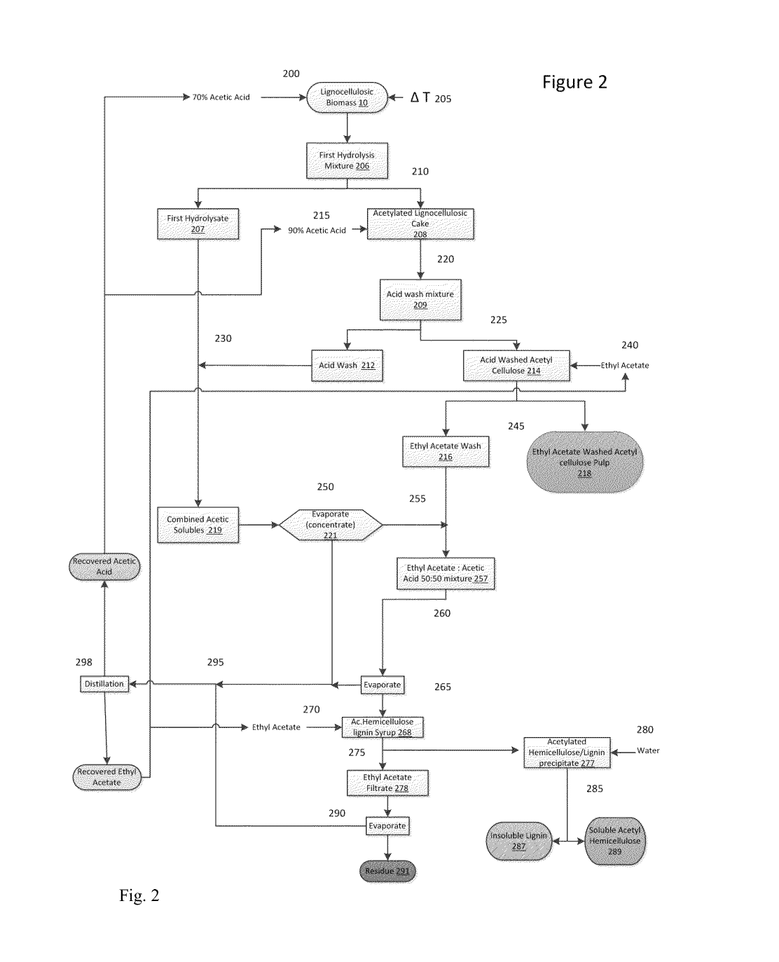

C₁-C₂ acid Hydrolysis. FIG. 2 illustrates one aspect of the invention pertaining to separation and recovery of a cellulose pulp, hemicellulose and lignin from a lignocellulosic biomass 10 utilizing a C₁-C₂ acid and C₁-C₂ acid-miscible organic solvent. The process is illustrated with acetic acid as the C₁-C₂ acid and ethyl acetate as the C₁-C₂ acid-miscible organic solvent as one preferred process, however, formic acid or mixtures of formic and acetic acid may also be used as substitutes for acetic acid and other C₁-C₂ acid miscible organic solvents may be used in combination with, or as substitutes for, ethyl acetate, provided that halogenated organic solvents are expressly excluded from certain embodiments.

A lignocellulosic biomass 10, illustrated and exemplified by corn stover, is mixed with the C₁-C₂ acid (acetic acid) at step 200. The final ratio of the C₁-C₂ acid to the lignocellulosic biomass should preferably be in the range of 3:1 to 5:1 on a wt:wt basis acid:dry solids, which excludes the water content of the C₁-C₂ acid and lignocellulosic biomass. Lower and higher ratios of C₁-C₂ acid to dry solids will work, but not as economically. The concentration of the C₁-C₂ acid to use is variable depending on the moisture content of the lignocellulosic biomass 10 so long as the aforementioned ratio of C1-C2 acid to dry solids is achieved. With a corn stover lignocellulosic biomass 10 dried to a moisture content of about 8%, 4.5 liters of 70% acetic acid per kilogram of biomass was adequate. When formic acid is used, the water content should be lower to achieve effective solubilization of lignin. Formic acid concentrations of 80-90% work well, whereas higher water content does not. Because acetic is more hydrophobic it tolerates more water to solubilize the same amount of lignin. At step 205 the acidified lignocellulosic biomass 10 is heated to a temperature and for a time sufficient to hydrolytically solubilize a first fraction of hemicellulose and lignin from the biomass 10 forming a first hydrolysis mixture 206. Preferably the heating 205 is done with agitation or with physical tumbling agents to apply mechanical force to the lignocellulosic biomass 10 during the heating and hydrolysis process 200/205. Optionally, in certain embodiments, the C₁-C₂ acid used in the initial hydrolysis 200/205 may be supplemented with no more than 0.25% to 1% w/v of a mineral acid such as HCl or sulfuric acid. The inclusion of small amounts mineral acid results in improved hydrolysis and solubilization of hemicellulose, however, also leads to a degradation of some of solubilized C5 sugars and to increased inorganic (ash) content, especially of the hemicellulose fraction that will be obtained. Further, if it is desirable to supplement the C₁-C₂ acid with sulfuric acid, it is additionally necessary to neutralize the sulfuric acid and to recover it as a sulfate salt. Residual sulfur is not compatible with certain catalyst that may be used for chemical conversion of sugars that may be desirable in certain biorefinery operations, and also may cause formation of sulfate esters that may interfere with subsequent enzymatic steps using cellulolytic, hemicellulolytic and esterase enzymes as described hereafter and in co-pending provisional application No. 61/538,211 entitled Cellulolytic Enzyme Compositions and Uses Thereof. Accordingly, in some embodiments sulfuric acid is specifically excluded from the acid hydrolysis steps 205 and 215.

Temperature and time conditions for hydrolytic release of hemicellulose and lignin are critical. If the temperature is too low or the time too short, there will be insufficient hydrolytic release of hemicellulose and lignin. Unexpectedly it was discovered that over-hydrolysis is detrimental to the recovery of useable materials. If the temperature is too high or the time is too long, unwanted hydrolysis of cellulose and hemicellulose to monosaccharides may occur and other reaction products will be formed that interfere with the subsequent precipitation of hemicellulose and lignin, leading to the formation of a gummy precipitate when reaction temperatures and/or times are excessive. The temperature should be in the range of 120-280° C. and the time should be in the range of 5-40 minutes. In a laboratory embodiment with 70% acetic acid, the temperature was raised to 165° C. in 10 minutes followed by quick reduction to a temperature of 150° C. over 3 minutes with gradual cooling thereafter to 100° C. over a 30 minute period. In an industrial plant embodiment, a temperature of 165° C. is used for a period of 1-10 minutes.

The first hydrolysis step 200/205 forms the first hydrolysis mixture 206 containing a soluble first hydrolysate 207 enriched in hemicellulose and lignin and an insoluble lignocellulosic residue fraction. At step 210 these are separated by a suitable technique such as filtration or centrifugation. The solid material is recovered as a first lignocellulosic cake 208 that is at least partially depleted of hemicellulose and lignin and that contains at least partially acylated cellulose (e.g., acetyl cellulose esters or formyl cellulose esters for acetic and formic acid, respectively). At step 215 the first recovered lignocellulosic cake 208 is thoroughly washed with the C₁-C₂ acid to further release bound hemicellulose and lignin. Preferably the C₁-C₂ acid used for the wash is warmed to a temperature of about 40-50° C. Optionally, but not necessarily, the acid wash of the first lignocellulosic cake 208 may include a second round of heat treatment using the same conditions of acid and heat as were used in the first round at steps 200/205 mentioned herein before. Whether or not the acid wash 215 should be done at elevated temperatures depends on the hemicellulose and lignin content and structure in the lignocellulosic biomass 10. When the lignocellulosic biomass 10 has a high lignin or hemicellulose content as in the case of woody sources, then a second round of heating 220 is preferred. The concentration of the C₁-C₂ acid is preferably higher at this wash step 215 than at hydrolysis step 200 because of dilution with water liberated by hydrolysis and from the water released by the lignocellulosic cake 208 from the initial treatment with the C₁-C₂ acid used at step 200. In the case of corn stover as the lignocellulosic biomass 10, 90% acetic acid was used in the acid wash step 215. The acid wash produces an acid wash mixture 209 that at step 225 is recovered by centrifugation or filtration into a liquid acid wash fraction 212 containing further hemicellulose and lignin separated away from the acid washed lignocellulosic cake 214, that has been depleted of a majority of the hemicellulose and lignin and which contains further acylated cellulose.

In a preferred process, at step 230 the first hydrolysate fraction 207 and the acid wash fraction 212 are mixed to form combined solution of acetic acid solubles 219. This combined acetic acid solubles solution 219 is then preferably evaporated at step 250 to a achieve a dissolved solids content of at least 30% wt/vol forming a concentrated soluble hydrolysate 221.

Separately, at step 240, the second lignocellulosic cake 214 is washed with ethyl acetate or other C₁-C₂ acid-miscible organic solvent to remove the C₁-C₂ acid and remaining hemicellulose and lignin from the second lignocellulosic cake 214. The total amount of the C₁-C₂ miscible organic solvent to use in washing 240 the second lignocellulosic cake 214 is preferably about the same quantity as the second amount of C₁-C₂ acid 215 used in the second hydrolysis step 220. The wash may be done with the total volume in batch, or preferably the total volume is applied in discrete increments to maximize removal of the C₁-C₂ acid and retained hemicellulose and lignin. The amount of acetic acid-miscible organic solvent to use for the wash should be sufficient to thoroughly wash the acetic acid from acetylated cellulose pulp. A total wash of at least 3 volumes (liters) of acetic acid-miscible organic solvent per weight (kg) of pulp is suitable. The total wash is preferably delivered in three or more discrete successive stages for delivery of the entire wash amount.

The wash results in a liquid organic solvent/C₁-C₂ acid wash fraction 216 that is separated at step 245 from the second lignocellulosic cake 214 by filtration. The filtration medium employed at step 245 should have pores large enough to permit passage of insoluble hemicellulose and lignin with the organic wash, yet small enough to retain the solid mass of higher molecular weight cellulose fibers in the acid washed cake 214 which after filtration is retained as organic solvent washed acyl-cellulose pulp 218. A suitable filtration medium for this filtration step 245 was one with pore sizes corresponding to a 60 mesh screen (nominal sieve diameter of 250 microns) or conventional filter paper.

At step 255 the organic solvent/C₁-C₂ acid wash fraction 216 is combined in roughly equal volumes with the concentrated hydrolysate 221 forming a C₁-C₂ acid/organic solvent mixture 257, which is agitated for a sufficient time to dissolve any insoluble hemicellulose and lignin obtained in the organic solvent wash 216. The C₁-C₂ acid/organic solvent mixture 257 is then evaporated at step 265 to a dissolved solids content of 40% wt/vol to form a concentrated hemicellulose and lignin syrup 268. A second amount of the C₁-C₂ miscible organic solvent is added to the concentrated hemicellulose and lignin syrup 268 in an amount sufficient to precipitate the hemicellulose and lignin. At a dissolved solids content of 40% with a mixture of acetic acid and ethyl acetate as the solvent system for the syrup 268, a ratio of 1 part syrup 268 to 3 to 4 parts ethyl acetate was sufficient to produce a filterable precipitate. At step 275 this hemicellulose and lignin precipitate 277 is separated from the syrup filtrate 278. Optionally, the hemicellulose and lignin precipitate 277 may be washed with further quantities of the C₁-C₂ acid-miscible organic solvent to remove residual C₁-C₂ acids.

It was discovered that the hemicellulose and lignin precipitate 277, if not further treated, may have residual acetate in the form of acetate salts (X-acetate). If it is desirable to reduce the acetate content of the recovered hemicellulose and lignin precipitate 277, then prior to precipitation, the concentrated hemicellulose and lignin syrup 268 may be titrated with a mineral acid, such as sulfuric acid, to adjust the pH to below the pKa of acetic acid—preferably to a pH of about 3.8. This exchanges the X of the acetate salt with the conjugate base of the mineral acid forming acetic acid, which along with the conjugate salt and base (e.g., X-sulfate), remains in solution when the hemicellulose and lignin are precipitated with ethyl acetate.

The hemicellulose and lignin precipitate is then mixed with warm water at step 280 to dissolve the hemicellulose forming a soluble hemicellulose aqueous fraction 289 and an insoluble lignin fraction 287, which are separated by filtration or centrifugation at step 285. Optionally, the insoluble lignin fraction 287 may be washed with a second round of warm water to extract more hemicellulose from the precipitate. Surprisingly, it was discovered that the temperature and cooling of the water used for solubilization of the hemicellulose and separation of the lignin from the precipitate is of critical importance. Heating the hemicellulose and lignin precipitate with water to 95° C. then cooling to 60° C. allowed the lignin to coalesce into larger particles which are much easier to filter and wash. In contrast, heating to 120° C. actually caused the lignin to form a solid mass which caused problems with handling and recovery of hemicellulose.

In the overall embodiment depicted in FIG. 2 the C₁-C₂ acid (acetic acid) and the C₁-C₂ acid-miscible organic solvent (ethyl acetate) is recovered from the process and recycled for continued use. Thus for example, the recovered ethyl acetate filtrate 278 is evaporated at step 290 to recover the ethyl acetate, leaving behind a dark residue 291. At step 295 the ethyl acetate and acetic acid recovered by evaporation at step 290 is combined with the acetic acid/ethyl acetate filtrate 261 and the acetic acid recovered from evaporation of the hydrolysate at step 250. These combined materials are then separated by distillation at step 298 to recover the acetic acid away from the ethyl acetate.

Almost all of the C₁-C₂ organic acid used in the process depicted in FIG. 2 is utilized in streams that can be readily separated by simple distillation from the C₁-C₂ acid miscible organic solvent rather than water. The combination of acetic acid and ethyl acetate were particularly effective. The C₁-C₂ acid-miscible solvents used in the process are chosen for their ability to precipitate both lignin and oligosaccharides as well as some monosaccharides from the C₁-C₂ acid. They also are easily separated from the organic acid by simple distillation. The processes of the prior art where acetic acid or formic acid are used in combination with water to separate hemicellulose and lignin from cellulose pulp suffer from the disadvantage of creating water acid azeotrope mixtures that are more difficult to recover and recycle for continued use. The processes of the present invention rely principally on the combination of the C₁-C₂ acid with a miscible organic solvent.

The processes described herein using the C₁-C₂ organic acid are advantageous they enable pulp and sugar fractions to be prepared from the preferred sources of lignocellulosic material, which are those from the monocot species, particularly the stalks of the cereal grains and bamboo. This is because monocot species have significantly higher silica content than dicot woods, which are the main source of cellulosic pulp in conventional processing. Monocot species are not used for conventional pulping to obtain a cellulose pulp, in-part because conventional pulping relies on sulfite treatment of woods. Sulfite treatment forms an undesirable solubilized silicate residue in processing streams that must be removed. The use of the C₁-C₂ organic acid, however, prevents solubilization of silicate residues because the silicates largely remain with the washed acetyl cellulose pulp 218. The process therefore provides an opportunity to make cellulose pulps 218 without reliance on sulfite treatment.

Compositional Analysis of the Soluble Hemicellulose Fraction.

A sample of the soluble hemicellulose fraction 289 obtained by the foregoing method was subjected to detailed chemical analysis for monomeric sugar, acid hydrolyzable sugar, lignin and acetic acid content, as well as other elemental substituents (see Table 21, Example 1). Of the total carbohydrates in the form of acid hydrolysable oligomers and monomeric sugars, about 19% were monomeric C5 and C6 sugars and about 81% were in the form of hydrolysable oligomers (hemicellulose oligomers). Together these accounted for about 68% of the total mass of the sample. The lignin content was only 0.28% of the mass. A small amount of acetic acid was retained through the process, accounting for about 1.2% of the mass. Most organisms used in fermentation to produce ethanol can tolerate up to 1% w/v acetic acid, but have a preference for concentrations well below 0.5% w/v at pH of around 6. If desired, the acetic acid content can be reduced by washing the hemicellulose/lignin precipitate 277 with ethyl acetate or other acetic acid miscible organic solvent prior to dissolving in water at step 280. In this case, it is preferable to use a less polar acetic acid miscible solvent, such as methyl ethyl ketone, propanol and the like so as to avoid removal of monomeric sugars from the soluble hemicellulose fraction 289.

From an exemplary practice of the forgoing, the mass distribution was as follows: From 1.5 kg of chopped corn stover at 92% solids content (1380 g starting solids material) about 810 grams was recovered in the ethyl acetate washed pulp 218, of which about 80% was in the form cellulose and which also contained about 10% pentoses. The concentrated ethyl acetate containing syrup 268 was about 50% dissolved solids and contained about 10% sugars and 60% lignin. From that, about 525 g of the starting solids material was recovered in the hemicellulose lignin precipitate fraction 277, of which about 45% was in the form of hemicellulose 289 and the remainder in the form of lignin 287.

Compositional Analysis of the Cellulosic Pulp

The cellulose and lignin content of the cellulose pulp 218 was analyzed by the ANKOM™ Fiber Analysis method (Vogel et al 1999) and the standard method defined by the National Renewable Energy Laboratory (NREL) Compositional analysis of lignocellulosic feedstocks. (Sluiter et al 2010). Analysis of several wet and dry fractions of the cellulose pulp 218 obtained from processing corn stover biomass 10 stover as described above is provided in Tables 1 and 2. The analysis by the ANKOM™ method (Table 1) indicates that cellulose represented 85.5 to 88.4% of the total dry matter with hemicellulose present in the range of 0.7-3.5% and lignin in the range of 1.0-2.3%. In samples treated with a combination of acetic and sulfuric acid, a higher concentration of cellulose was obtained with increased sulfuric while the hemicellulose is reduced, consistent with greater hydrolysis of hemicellulose. By comparison, the samples analyzed with the NREL method (Table 2) indicate the presence of a lower cellulose concentration in the range of 62.2-77.3%, while hemicellulose and lignin were higher by this method (3.2-15.8% and 1.0-5.8%). When the samples are treated with a combination of acetic acid and sulfuric, an increase in cellulose content with a parallel reduction in hemicellulose was also observed. While analyses by the ANKOM™ method indicated some variability in the pulp and a lower content in the overall cellulose composition when compared with the NREL method, material balance measurements indicated consistent accounting for the majority of the solids by both methods (range 94.1-108.8% with an average of 99.1%).

TABLE 1
Compositional Analysis of Cellulosic pulp 218
by ANKOM ™ Fiber Analysis Method

| | | Cellu- | Hemi- | |
|---|---|---|---|---|
| | | lose | cellulose | Lignin |
| | | % Dry | % Dry | % Dry |
| Sample | Description | Matter | Matter | Matter |
| Sample 1A | Wet Stover pulp Cake Sam- | 85.50 | 1.11 | 1.54 |
| | ple A | | | |
| Sample 1B | Wet Stover Cake Pulp Sam- | 88.41 | 2.89 | 1.04 |
| | ple B Sa | | | |
| Sample 1A.d | Dried Corn Stover Pulp | 85.33 | 3.53 | 1.87 |
| | Sample A | | | |
| Sample 1B.d | Dried Corn Stover Pulp | 88.02 | 0.41 | 1.45 |
| | Sample B | | | |
| Wet Cake .A | Wet Cake (70% AcOH w/ | 85.27 | 2.50 | 2.30 |
| | 0.25% H₂SO₄) | | | |
| Wet Cake B | Wet Cake (50% AcOH w/ | 87.91 | 0.71 | 1.83 |
| | 0.5% H₂SO₄) | | | |

TABLE 2
Compositional Analysis of Cellulosic Pulp 218 by the NREL Method

| | % | % | % | % | % | % | % | % | Total |
|---|---|---|---|---|---|---|---|---|---|
| Description | Ash | Protein | Lignin | Glucan | Xylan | Galactan | Arabinan | Acetate | % |
| Sample 1A.d | 6.45 | 1.75 | 4.56 | 78.85 | 12.64 | 0.68 | 0.78 | 3.09 | 108.80 |
| Sample 2 | 5.67 | 0.63 | 3.01 | 68.91 | 15.84 | 0.79 | 0.87 | 2.33 | 98.04 |
| Sample 3 | 8.16 | 1.19 | 2.74 | 72.11 | 10.11 | 0.71 | 0.61 | 2.65 | 98.27 |
| Sample 4 | 9.77 | 1.17 | 4.49 | 77.26 | 3.23 | 0.68 | 0.61 | 3.26 | 100.47 |
| Sample 5 | 8.14 | 2.73 | 0.95 | 71.64 | 10.77 | 0.60 | 0.50 | 2.64 | 97.96 |
| Sample 6 | 16.21 | 3.61 | 0.12 | 62.20 | 8.35 | 0.46 | 0.91 | 2.23 | 94.09 |
| Sample 7 | 11.58 | 0.81 | 3.74 | 71.50 | 6.43 | 0.52 | 0.62 | 2.44 | 97.64 |
| Sample 8 | 7.04 | 1.50 | 5.82 | 67.26 | 12.02 | 0.44 | 1.03 | 2.35 | 97.47 |

The cellulose pulp 218 was further examined to characterize the cellulose fibers present therein for their utility as a substitute or complement for cellulose fibers made from typical paper pulping operations that rely on sulfite treatment of lignocellulosic woods. An acylated cellulose pulp 218 was made from corn stover, wheat straw, and two types of bamboo (Moso, and Henon). Table 2B below summarizes some of the pertinent characteristics of the pulp fibers obtained.

TABLE 2B
Fiber Characteristics of Acetylated Cellulose Pulps

| | | | Moso | Henon |
|---|---|---|---|---|
| | Stover | Wheat | Bamboo | Bamboo |
| Average Length | 1.12 | 1.14 | 1.3 | 1.61 |
| Weighted Fiber | | | | |
| Length (LWFL) | | | | |
| Coarseness/LWFL | n/a | n/a | 8.9 | 8 |
| Unrefined Canadian | 507 | 462 | 635 | 689 |
| Standard Freeness | | | | |
| Britt Jar Fines | 7.6 (13.8) | 8.0 (14.9) | 28% | 30% |
| Tensile Index @ 450 | 28 | 23 | 23.1 | 20.9 |
| CSF | | | | |
| Tear Index/Tensile | 0.18 | 0.16 | 0.14 | 0.1 |
| Index | | | | |
| Burst Index/Tensile | 0.053 | 0.054 | 0.048 | 0.048 |
| Index | | | | |

(X.X) = Fines of homogenous pulp

The above data indicate that the acetylated cellulose pulp fibers made from monocot species according to the methods provided herein are suitable as substitutes and/or as amendments for fibers made from conventional wood pulping.

Treatment of the Soluble Hemicellulose 289 or the Cellulose Pulp 218 to Make a C6 or C5 Enriched Syrup.

The cellulose pulp 218 is primarily cellulose (62.2% to 88.4% by weight depending on method of analysis and sample analyzed), which when digested by a suitable cellulolytic enzyme cocktail will produce a syrup enriched with C6 sugars—primarily glucose. The solubilized hemicellulose enriched fraction 289 is a hemicellulose stream nearly devoid of lignin and is made up of a mixture of monomers and oligomers of xylose with traces of arabinose, glucose, and other hexose sugars. When fully digested by a suitable hemicellulolytic enzyme cocktail the soluble hemicellulose enriched fraction 289 will produce a syrup primarily enriched in C5 sugars. The terms “cellulolytic enzyme” and “hemicellulolytic enzyme” and cocktails thereof, means one or more (e.g., “several”) enzymes that hydrolyze a cellulose or hemicellulose containing material, respectively. Examples of such enzymes are provided in pending U.S. provisional application No. 61/538,211 entitled Cellulolytic Enzyme Compositions and Uses Thereof. It was discovered by the present applicants, however, that conventional cellulolytic and hemicellulolytic enzyme cocktails available for digestion of cellulose and hemicellulose, did not operate efficiently with the cellulose pulp and soluble hemicellulose fractions prepared by acetic acid treatment of corn stover as the lignocellulosic biomass. Initial results showed that the enzymatic hydrolysis of the solubilized C5 syrup and the C6 pulp proceeded slowly, even with high enzyme loading, and the amount of monosaccharides released was less than predicted.

Initial Hemicellulose 289 Hydrolysis.

Enzyme hydrolysis of the soluble hemicellulose fraction 289 was carried out to convert the soluble hemicellulose oligomers to monomers for fermentation. Total carbohydrate analysis of this fraction by the phenol-sulfuric acid method indicated a total carbohydrate concentration of 65% w/w dry mass basis. The initial enzyme hydrolysis employed cocktails of commercial enzymes available from Novozymes A/S (Bagsvaard, Denmark) under the trade names Cellic CTec (cellulase(s)) and Viscozyme L (pectinase(s)) blended in a 4:1 ratio was used at an enzyme dose rate of 2% w/w dry basis of soluble hemicellulose 289 solids diluted to 10% wt/vol with 50 mM citrate buffer pH 5.0. Samples were incubated at 50° C. for five days. Results are provided in Table 3 below. These indicate a yield of 82.7% of monomers of the total carbohydrates after enzyme hydrolysis. Only about 80% of the total carbohydrates were in the form of acid hydrolysable hemicellulose oligomers, so the percentage of hemicellulose oligomers converted to monomeric sugars was only about 65%.

TABLE 3
Results of Enzyme Hydrolysis of Hemicellulose from Corn Stover

| | C5 Fraction | | |
|---|---|---|---|
| | Dextrose | Xylose | Arabinose |
| | %, db | %, db | %, db |
| Enzyme Hydrolyzed | 13 | 37.4 | 3.4 |
| Control | 6.3 | 18.2 | 3.5 |

Initial Cellulose Pulp 218 Hydrolysis

Enzymatic hydrolysis of the cellulose pulp 218 prepared as described above was also conducted. A cocktail of two commercial enzymes from Novozymes (Cellic CTec2 (cellulase(s)) Cellic HTec2 (xylanase(s)) were used for the enzyme hydrolysis of the cellulose pulp fraction. Other commercial and non-commercial enzyme blends were also tested. The cellulose pulp 218 analyzed by the ANKOM™ fiber analysis method averaged 86.7%, 1.8%, and 1.7% on a w/w dry basis cellulose, hemicellulose, and lignin, respectively, for six samples of cellulose pulp 218. Bench scale enzyme hydrolysis was carried out at both low solids (10%) and high solids loading (20%). Low solids enzyme hydrolysis produced 87.8% conversion of the cellulose pulp 218 to glucose and xylose, whereas, high solids enzyme hydrolysis experiments with 20% dry solids loading gave over 82.6% conversion to glucose and xylose (Table 4). Enzyme hydrolysis in both experiments was carried out at 50° C. for five days. The enzyme dose for low solids enzyme hydrolysis was 12 mg enzyme protein/g pulp dry solids. For high solids enzyme hydrolysis the dose was 33 mg enzyme protein/g pulp dry solids.

TABLE 4
Enzyme Hydrolysis of Cellulose Pulp 218 Obtained
from Corn Stover at 10-20% Dry Solids

| | | | | | Enzyme |
|---|---|---|---|---|---|
| | Initial Dry | | | Acetic | Hydrolysate |
| | Solids | Dextrose | Xylose | acid | Dry Solids |
| | g/kg | g/kg | g/kg | g/kg | g/kg |
| 10% solids | 100 | 75.0 | 10.5 | 2.0 | 108 |
| 20% solids | 200 | 130.8 | 31.6 | 0.7 | 217.1 |

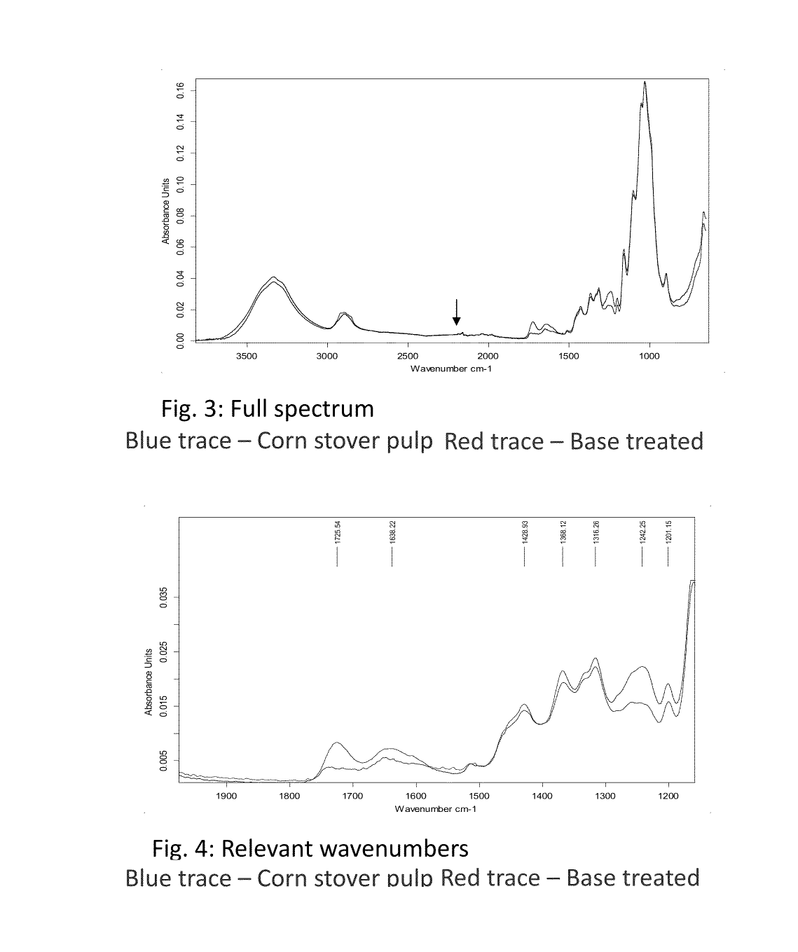

The foregoing results showed less conversion of the soluble hemicellulose fraction 289 and the cellulose pulp fraction 218 to monomeric sugars than is needed to make subsequent fermentation economically practical. These materials were made by exposure of the biomass to heat in the presence of high concentrations of acetic acid (>70%). It was speculated that some of the free and bound sugars may have become substituted with acetyl groups and that this acetylation may at least partially inhibit enzymatic activity. To test this, samples of the cellulose pulp 218 were treated with a base to catalyze deesterification of the acetate group. The result was assessed by Fourier Transform Infrared Spectroscopy (FTIR). FIG. 3 illustrates the difference in FTIR spectra of corn stover cellulose pulp (top trace) and ammonium hydroxide treated corn stover pulp (lower trace). FIG. 4 shows the FTIR spectra between 1150 cm⁻¹ and 2000 cm⁻¹, where three important ester bonds are represented by the C═O ester stretching at 1725 cm⁻¹, the C—H stretching in —O(C═O)—CH₃ group at 1366 cm⁻¹, and the —CO— stretching of acetyl group at 1242 cm⁻¹ are indicated. The absence of a peak at 1700 cm⁻¹ representing the absorption of a carboxylic group confirmed that the alkaline treated sample is free of esterified acetic acid.

It was this result that indicated that acetic acid hydrolysis of lignocellulosic biomass 10 according to FIG. 2 resulted in a cellulose pulp 218 that was acetylated. More generally, treatment of a lignocellulosic biomass 10 by a C₁-C₂ acid results in a significant fraction of the cellulose pulp 218 as well as the soluble hemicellulose fraction 289 being acylated by the C₁-C₂ acid hydrolysis 210 and wash steps 220 (i.e., the carbohydrate fractions will contain formyl- or acetyl-esters). Hence, production of suitable feedstock C5 and C6 sugar syrups for fermentation by enzyme digestion requires deacylation of the esters prior to, or in conjunction with, digestion of the cellulose polymers or hemicellulose oligomers with the appropriate enzyme cocktails.

Formylated carbohydrate esters made when the C₁-C₂ acid is formic acid are heat labile. Accordingly, a formylated cellulose pulp 218 or soluble hemicellulose fraction 289 can be deformylated by incubation of the material in an aqueous solution at a temperature of 50° C. to 95° C. for 0.5 to 4 hours, which is sufficient to deformylate the carbohydrates as described for example in Chempolis, U.S. Pat. No. 6,252,109. Acetylated carbohydrates, however, are more stable than formylated esters. Acetyl esters can be deacetylated by treatment with an alkali (base). Suitable bases include ammonia (ammonium hydroxide) and caustic (sodium hydroxide). Accordingly, the cellulose pulp 218 and soluble hemicellulose fractions were treated by contact with alkaline bases prior to enzymatic digestions. Acetic acid treated corn stover pulp sample preparations 218 were diluted with water to form a mixture of 8% solid. NaOH was added to adjust the pH to 13. The mix was heated to boiling, and kept boiling for 10 min. Phosphoric acid was used to adjust the pH to 5.0 after the reaction mix reached room temperature. A control cellulose pulp 218 was heated similarly at the same time and at the same solid content without sodium hydroxide treatment or pH adjustment. The alkali treated samples were adjusted to a 5% dissolved solids mixture and analyzed for acetic acid with the results shown in Table 5.

TABLE 5
Deacetylation of Cellulose Pulp 218 by Base

| | acetic acid (mg/g) |
|---|---|
| NaOH treated | 1.68 |
| Control | 0.75 |

The results indicated that more acetic acid was freed by the alkaline treatment as compared with the untreated control. The acetic acid that was freed by the heated alkaline treatment provided additional confirmation that acetyl groups are covalently linked to carbohydrate pulp fiber molecules via ester links formed during the acetic acid treatment steps. The degree of esterification in various cellulose pulp 218 fractions made by the processes described herein ranged from a 0.05 to 0.2 degree of substitution (i.e., 5%-20% of the sugar residues are acetylated) which corresponds to 1.4% to 6.6% w/w acetyl content of the mass of the cellulose pulp fraction.

To confirm whether the deesterification would improve enzyme digestibility the treated cellulose pulp 218 samples prepared above were subjected to enzyme hydrolysis at 5% solids content with citrate buffer and a commercial cellulase enzyme blend from Novozymes (Cellic Ctec). Enzyme treatments were carried out in a rotisserie incubator (Daigger FinePCR Combi D24) at 50° C. for 96 hrs. The enzyme treated samples were analyzed for sugars by HPLC. Table 6 provides a summary of analytical results.

TABLE 6
Impact of Base Treatment on Enzymatic Release
of Glucose from Corn Stover Cellulose Pulp 218

| Content mg/g | Glucose | Xylose |
|---|---|---|
| NaOH treated | 12.8 | 4.0 |
| Control | 6.1 | 3.0 |

The results indicated that enzyme treatment of alkaline diesterified cellulose pulp 218, results in a substantially higher release of glucose and xylose. The results further supported the finding that acetate esters hindered enzyme access to cellulose in the aforementioned enzymatic digestions using different mixtures of cellulolytic and hemicellulolytic enzymes. Presumably by removing the acetate esters, the enzymes can access and bind the substrate better and therefore, hydrolyze more cellulose pulp 218 and hemicellulose 289 fiber polymers, resulting in release of more glucose and other monomeric sugars. The results also indicate that heating 10 to 30 minutes in an autoclave at 121° C., with ammonia at a concentration of 0.1% to 1%, or at the lower temperature of 50° C. for 1 to 10 h, with ammonia 0.5 to 5% is sufficient to release most acetyl groups from the pulp.

Detergents

It was further discovered that non-ionic detergents can substantially increase the activity of hemicellulolytic and cellulolytic enzyme preparations. Cellulose pulp samples 218 were treated with alkaline NaOH followed by treatment with a commercial enzyme cellulase blend. Many detergent chemicals, including Tween-20 (polyoxyethylene sorbitan monolaurate), Tween-40 (polyoxyethylene sorbitan monopalmitate), Tween-60 (polyoxyethylene sorbitan monostearate) and triton X-100 (4-octylphenol polyethoxylate) were tested to determine their function on enzyme hydrolysis of the pulp. The enzyme reaction contained 5% pulp solids wt/wt of a 50 mM citrate buffer, the commercial cellulase enzyme blend Cellic Ctec II, with or without detergents, for example, Tween-40 at 0.2% w/w content. After 6 days, the resulting mixtures were analyzed for glucose by HPLC.

TABLE 7
Impact of Tween 40 on Release of
Glucose from Cellulose Pulp 218

| Additive | glucose (mg/ml) |
|---|---|
| Tween-40 Supplementation | 38 |
| Control without addition | 20 |

In another test, cellulosic pulp 218 from acetic acid treated corn stover prepared as described herein but not deacetylated by base treatment was dried and treated with Novozymes' cellulase blend Cellic CTec2, Novozymes pectinase Viscozyme L or xylanase Htec2 hemicellulase blends, at high and low enzyme doses, with or without Tween 40. The results provided in Table 8, indicate that Viscozyme consistently released more sugar than HTec2, and importantly, that including Tween 40 in the treatment step, resulted in a higher release of sugar event when the cellulose pulp 218 was not deacetylated. The results also indicated that high enzyme dose can at least partially overcome inhibition of the cellulases by acetylation of the cellulosic pulp during pretreatment. This further suggest that the tested cellulase enzyme blends have a low level of esterase activity that is present and that including more esterase activity in the blend can be useful in reducing cellulase enzyme loading.

TABLE 8
Improved Glucose Release from Cellulose Pulp With Cellulases/Tween 40

| Sample | Tween 40 | Total Enz | | Dextrose (g/L) by HPLC | | | | | |
|---|---|---|---|---|---|---|---|---|---|
| ID | (0.02% w/w) | (%, v/db) | Enz 2 | Day 3 | Day 6 | Day 9 | Day 13 | Day 16 | Day 20 |
| 1 | Yes | 4.5 | Viscozyme L | NL | NL | NL | 109.0 | 121.2 | 128.4 |
| 2 | No | 4.5 | Viscozyme L | NL | NL | NL | NL | NL | NL |
| 3 | Yes | 4.5 | Htec2 | NL | NL | NL | NL | 103.7 | 119.6 |
| 4 | No | 4.5 | Htec2 | NL | NL | NL | NL | NL | NL |
| 5 | Yes | 25 | Viscozyme L | NL | 147.0 | 153.0 | 153.1 | NS | NS |
| 6 | No | 25 | Viscozyme L | NL | 115.1 | 129.0 | 124.3 | NS | NS |
| 7 | Yes | 25 | Htec2 | NL | 133.6 | 149.5 | 152.7 | NS | NS |
| 8 | No | 25 | Htec2 | NL | 107.0 | 121.3 | 129.2 | NS | NS |

The results summarized in Tables 7 and 8 indicate that the addition of detergent to a variety of cellulolytic and hemicellulolytic enzyme reactions results in a substantially greater release of glucose as compared with the control treated sample without the addition of Tween 40. Other non-ionic detergents that may also be suitable for enhancing the enzymatic activity of cellulolytic and hemicellulolytic enzyme preparations include, but are not limited to Tween-20, Tween-60, Tween-80 and Triton X-100. The amount of detergent to use should range from 0.01% and 5% v/wt of the reaction mix.

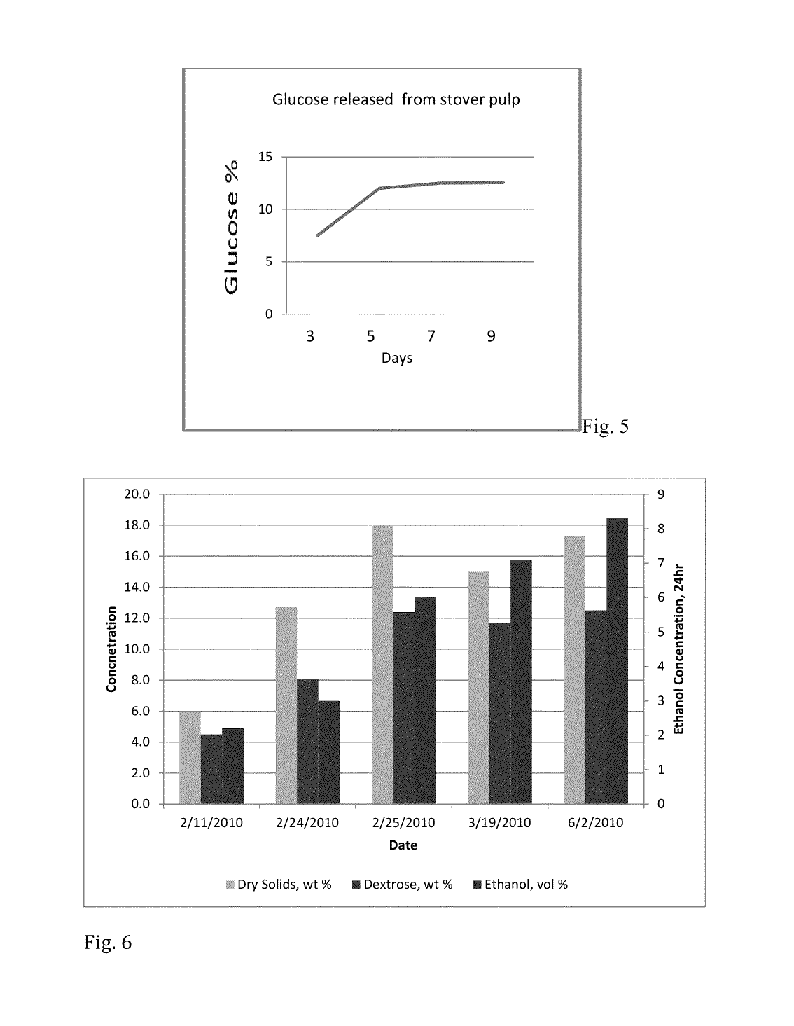

In another test the acetylated cellulose pulp 218 obtained after ethyl acetate washing was washed extensively with water after filtration to remove any free acetic acid. To the washed sample, NH₄OH was added to a final concentration of 0.5% (v/w). The samples were treated at 121° C. for 30 min to deacetylate the sample. Phosphoric acid, buffer and commercial enzymes (dosed at 3% of the DS) and Tween-40 (added to 0.5% w/v) were added to the base treated samples to make a 15% solids reaction mix. The samples were placed in a 50° C. incubator and rotated at 20 rpm. After 2 days of incubation, the cellulose pulp 215 started to liquefy. On the third day, the glucose content was measured. Additional samples were removed daily afterwards to check for glucose. The glucose released by the enzyme reaction is graphed in FIG. 5. After 7 days of incubation at 50° C., most of the glucose estimated to be present in the cellulose pulp 218 was released. The composition of the hydrolysate after 9 days was (on a w/w (Dissolved Materials basis) glucose 12.56% (84% DM), xylose 1.73% (11.5% DM), ash 2.0% (13.3% DM) and acetic 0.56% (3.7% DM).

Aliquots of the 9-day enzyme treated hydrolysate, were fermented by different yeast strains at 30° C. in stoppered shaker flasks rotated at 150 rpm. The culture was inoculated at a pitching rate of 250 million cells/ml. Samples were taken during fermentation at 24 hr and 48 hr. These samples were analyzed for sugars, organic acids and ethanol. The results indicate that one of the strains of yeast tested that was engineered to utilize xylose for fermentation (namely S. cerevisiae 424a, available from Purdue Research Foundation, Lafayette, Ind.) produced 5.6% ethanol (v/v) in 24 hr and used 50% of xylose within 48 hr.

Incorporation of Supplemental Esterases

Although as described herein above, base catalyzed deesterification of the acylated cellulose pulp 218 and hemicellulose fractions 289 improves enzyme digestibility, it requires extra materials and produces a basic reaction mixture that must be pH adjusted before enzymatic digestion of the cellulose pulp 218 and soluble hemicellulose 289 fractions. It was surprisingly discovered, however, that these fractions can also be efficiently deacetylated by co-treatment with an esterase enzyme. This discovery was based in part on analysis of released acetic acid when a cellulose pulp 218 was treated with a cocktail of commercial hemicellulases and cellulases from Novozymes (Cellic CTec2 and HTec2). Such enzyme preparations are not highly purified to obtain one protein with one specific type of enzymatic activity but rather are cocktails of various partially purified enzyme activities that contain residual activities of other enzymes that co-purify in the preparation process. At high enzyme loading, some de-acetylation of the cellulose pulp 218 was observed consistent with a low level of esterase enzyme type activity being present in the enzyme blend. This formed the basis of seeking to incorporate more esterase activity by adding additional esterase activities preparations to cocktails of cellulolytic and hemicellulolytic enzyme preparations.

A suitable esterase for making the C6 and C5 syrups made from C₁-C₂ acid treatment of the cellulose pulp and hemicellulose fractions made as described herein should display at least one activity that catalyzes the hydrolysis of acetyl groups from at least one of: a polymeric xylan, acetylated xylose, acetylated glucose, acetylated cellulose, and acetylated arabinose. Co-pending U.S. patent application No. 61/538,211 entitled Cellulolytic Enzyme Compositions and Uses Thereof describes at least one example of such an esterase denoted acetylxylan esterase (AXE) that can be used to accomplish improved digestion of the cellulosic pulp 218 and soluble hemicellulose fractions 289 made as described herein to provide improved C6 and C5 syrups. AXE is a carboxylxylesterase (EC. 3.1.1.72) that catalyzes the hydrolysis of acetyl groups from polymeric xylan, acetylated xylose, acetylated glucose, alpha-napthyl acetate and p-nitrophenylacetate. Its activity is measured by deacetylation of p-nitrophenylacetate in acetate buffer at pH 5.0, which provides the colorimetric product p-nitorphenolate. One unit of AXE is defined the amount of enzyme that releases 1 μmole of p-nitorphenolate per minute at 25° C. Co-pending U.S. patent application No. 61/538,211 entitled Cellulolytic Enzyme Compositions and Uses Thereof provides further data demonstrating that incorporation of such an esterase activity for digestion of the pulp 218 and soluble hemicellulose fractions 289 described herein improves conversion of the material to C6 and C5 syrups.

Yet another example of a suitable esterase is represented by a GDSL hydrolase/acetylxylan esterase from Geobacillus stearothermophilus described by Alalouf O, Balazs Y, Volkinshtein M, Grimpel Y, Shoham G, and Shoham Y. (J Biol Chem. 2011, 286(49):41993-2000) which is categorized as a carbohydrate esterase (CE) in the CE 17 family.

Still other suitable examples include, any number of the carbohydrate esterases grouped into the CE 1-7 families as summarized by Peter Biely in Microbial Carbohydrate Esterases Deacetylating Plant polysaccharides, Biotechnology Advances, 2012 online publication, incorporated herein by reference. These include acetylxylan esterases (AcXEs), acetyl esterases (AcE), chitin deacetylases, peptidoglycan deacetylases, feruloyl esterases, pectin acetyl esterases, pectin methylesterases, glucuronoyl esterases and enzymes catalyzing N-deacetylation of low molecular mass amino sugar derivatives. The CE1 family are serine esterase with a SHD triad structure of active site amino acids that are AcXEs active also on acetylgalactoglucomannan and acetylated carbohydrates, including hexosides; and on cellulose acetate. The CE 2 family are serine esterases with a SH diad structure of active site amino acids that are 6-O-deacetylases of hexopyranosyl residues of poly- and oligosaccharides; with specific activity on acetylxylan and xylosides that is low; but catalyze transesterification exclusively to position 6 of hexoses and hexosyl residues. The CE 3 family are serine esterase with a SHD triad structure of active site amino acids that are AcEs with wide substrate specificity and form and usually are a component of bifunctional enzymes. The CE 4 family are metallo enzymes with aspartic acid in the active site that are specific AcXEs, not active on acetylgalactoglucomannan and acetylated manno-compounds but are active on chitin and in peptidoglycan N-deacetylation. The CE 5 family are serine esterase with a SHD triad structure of active site amino acids that are AcXEs deacetylating the 2 position of xylopyranosyl residues in acetylxylan and xylooligosccharides and glycosides; and which deacetylate mannosides and cellulose acetate. The CE 5 family are serine esterase with a SHD triad structure of active site amino acids that are AcXEs deacetylating acetylxylan but which have a broader substrate specificity. The CE 7 family are serine esterase with a SHD triad structure of active site amino acids that are active in deacetylating oligosaccharides transported into cells and also known as cephalosporin C deacetylases. The active site amino acids of the CE 16 family are not known, but these enzymes are exo-acting xylooligosaccharide deacetylases that deacetylates only the nonreducing-end sugar residues, appear to be inactive on polymeric substrate, and catalyze transesterification to position 3 of glycosides and the non-reducing sugar.

Some more specific but no limiting examples of suitable acetyl esterases from these families with their abbreviated enzyme name and CE family include: Schizophyllum commune ScCE1 (AcXE); Penicillium purpurogenum PpCE1 (AcXEI); Penicillium purpurogenum PpCE5 (AcXEII); Celvibrio japonicus CjCE2 (AcF); Streptomyces lividans SlCE4 (AcXE); Clostridium thermocellum CtCE4 (AcXE); Trichoderma reesei TrCES (AcXEI); Orpinomyces sp OxCE6 (AcXE); Thermotoga maritime TmCE7 (AcE); and Bacillus pumilus BpCE7 (AcE).

Fermentations

The preparations of soluble hemicellulose 289 and the hemicellulose and lignin depleted cellulose pulp 218 materials made according to the processes described herein are used to make C5 and C6 sugars suitable as feedstocks for microorganisms employed to make a variety of products by fermentation. A variety of protocols for utilization such materials are possible, depending on the organism employed and the fermentation product being made. Most microorganisms can utilize the palette of C5 and C6 sugars made by digestion of these material as a carbon source for cell growth (biomass accumulation). Biomass accumulation, however, is only one factor pertinent to the economics of production of the final fermentation product. For example, while a variety of yeast can utilize C5 sugars for biomass accumulation under aerobic growth conditions, most yeast do not produce ethanol by fermentation under such conditions. Conversely, under anaerobic conditions where yeast do produce ethanol from glucose and other C6 sugars, Saccharomyces yeast do not have the metabolic pathways necessary to divert the C5 sugars D-xylose and L-arabinose into ethanol production, unless they have been genetically engineered with exogenous enzyme activities to divert the C5 sugars into to the glycolytic pathway. In contrast, genetically engineered strains of the bacterium Zymomonas mobilis, have the capacity to produce ethanol by fermentation on either C5 or C6 sugars under anaerobic conditions. Still Zymomonas, like yeast and most other microorganisms show a preference for the uptake of glucose first before the uptake of other C6 or C5 sugars.

Several variations for digestion and fermentation of the C5 and C6 sugars produced form the hemicellulose 289 and cellulose pulp 218 made by the methods provided herein are possible. In one embodiment, the hemicellulose 289 and cellulose pulp 218 are first separately digested with enzymes to form separate C5 and C6 sugars. Subsequently, these feedstocks are fed to the microorganism to produce the fermentation product. When enzymatic digestion is conducted separately from subsequent fermentation to create a syrup, this is referred to as separate hydrolysis and fermentation with the abbreviation SHF.

In a SHF process the hemicellulose fraction 289 made by the processes of the invention is digested with appropriate enzyme cocktail containing cellulase, hemicellulase, pectinase, esterase and optionally protease activities at temperatures of up to 70° C. and pH of 4.0-6.0 with continuous mixing to yield a C5 enriched sugar syrup. In the preferred embodiment, the enzyme digestions of the hemicellulose fraction 289 are carried out at 50-65° C. at a pH of 5.0 for 1 to 7 days. To yield the greatest amount of sugars, the enzyme digestion reaction mixtures also contain a non-ionic detergent such as Tween 40 as discussed herein above. Using the detergent allows the solids content of the cellulose pulp 218 or soluble hemicellulose fraction to be in range of 10%-25% w/w. The C5 sugar syrup resulting from the digestions is then either directly used as a feedstock in the fermentation media to either accumulate biomass, or to accumulate biomass and produce the desired fermentation product.

Similarly, the cellulose pulp 218 made as described herein can be subjected to enzyme digestion after suspending in an aqueous buffer solution at a pH of 4.5-5.5 at 10-25% dry solids using a cellulase blend of enzymes including an esterase at a temperature of 50° C. for 5 days to yield a fermentation feedstock comprised of the C6 sugar enriched syrup. Again, a non-ionic detergent such as Tween 40 is included in the digestion mixture which permits use of the high solids content of 10-25% cellulose pulp to maximize the yield of the C6 sugars.

If the desired fermentation product is ethanol and the fermenting microorganism is a genetically engineered strain of S. cerevisiae, the yeast is grown on the C5 sugar syrup alone under anaerobic conditions for a time sufficient to accumulate biomass and first portion of ethanol in a first stage. In a second stage, the fermentation broth is supplemented with a C6 sugar source, preferably glucose, or sucrose, or mixtures of the same, and the fermentation is continued under anaerobic conditions for a time sufficient to accumulate a second portion of ethanol. The C6 sugar source may include the C6 syrup prepared from the cellulose pulp 218 as described herein.

A SHF process to ferment ethanol was done using the C6 syrup obtained from digesting the cellulose pulp 218 at high enzyme high solids (20%) described in Table 4 above. A number of commercial and non-commercial strains were tested including xylose engineered recombinant strains of S. cerevisiae capable of fermenting C5 sugars to make ethanol. The strains tested include an in-house Saccharomyces cerevisiae production strain Y500 (Archer Daniels Midland Company, Decatur, Ill.) an in-house engineered strain capable of D-xylose fermentation designated 134-12 that is derived from Y-500, a commercial strain obtained from the Fermentis division of the LeSaffre Group (Milwaukee, Wis.) designated ER2, and a GMO strain of Saccharomyces cerevisiae engineered for xylose fermentation by Nancy Ho of Purdue University (Purdue Research Foundation, West Lafayette, Ind.) that is designated 424a. For the initial bench scale experiments, separate saccharification and fermentation trials were run to determine fermentation capacity using the xylose engineered recombinant strain 424a, which was described in Sedlak et al Enz. Microbial Technol. 33, 19-28 (2003). Table 9 shows the consumption of glucose (dextrose) and xylose and concomitant production of 8.5% v/v yield of ethanol in a 48 hour period using C6 and C5 syrups from deacetylated corn stover pulp.

TABLE 9
Production of Ethanol from C6 Syrup from
Acetic Acid treated Cellulose Pulp 218

| | Dex- | Xy- | Lactic | Glyc- | Acetic | Eth- |
|---|---|---|---|---|---|---|
| Time | trose | lose | acid | erol | acid | anol |
| hours | g/L | g/L | g/L | g/L | g/L | %, v/v |
| 0 | 146.9 | 25.9 | 0 | 12 | 1.0 | 0 |
| 24 | 0.5 | 11.3 | 0 | 13.5 | 1.2 | 6.3 |
| 42 | 0 | 9.4 | 0 | 17.8 | 2.6 | 8.5 |

Nearly all of the dextrose and 56% of the xylose was consumed in the first 24 hours.

Further studies of SHF processes to ferment ethanol were carried out using the C6 syrup obtained from digesting the corn stover cellulose pulp 218 to produce an ethanol solution that will be economical to recover by distillation. Economical distillation is normally attained with at least 6.5% ethanol, which suggests that sugar solutions needed to attain this concentration need to be around 10%. Sugar solutions from enzyme hydrolysis at 10% by weight further suggest that enzyme hydrolysis must be carried out at high solids loading, between 15-20% by weight. High solids enzyme hydrolysis presents several problems, such as inadequate mixing, heat transfer and high viscosities. Several strategies were attempted to produce a concentrated sugar solution from enzyme hydrolysis, including low solids enzyme hydrolysis coupled to evaporative concentration, processive addition of solids during low solids enzyme hydrolysis and ultimately high solids enzyme hydrolysis with surfactant addition. Initial experiments resulted in 2.2% v/v ethanol from the fermentation of low solids (6%) enzyme hydrolysis of cellulose pulp 218 with two fold evaporative concentration. (Table 10) Subsequent experiments produced 3% v/v ethanol from the fermentation of material produced by the enzyme hydrolysis of 9% solids cellulose pulp 218 with addition of cellulose pulp 218 to 14% total solids after solubilization of the initial solids. (Table 11) Ethanol was produced at 6% v/v concentration from material that was evaporatively concentrated two fold from enzyme hydrolysis of 9% solids cellulose pulp 218. (Table 12) Evaporative concentration adds an expensive step to commercial production of ethanol, so the alternative of high solids enzyme hydrolysis of cellulose pulp 218 with surfactant addition was tested. Several yeast strains produced ethanol from 6.8-7.1% v/v during shake flask fermentation of high solids enzyme hydrolysis of 16.5% solids cellulose pulp 218 with surfactant addition. (Table 13) Finally, material produced by high solids enzyme hydrolysis at 20% solids cellulose pulp 218 with surfactant addition was fermented in shake flasks by yeast strain 424a and produced 8.3% v/v ethanol. (Table 14) A graphical summary of the data, including pulp dry solids, sugar concentration and ethanol concentration produced, from Tables 10-14 is presented in FIG. 6.

TABLE 10
Shake Flask Fermentation of C6 Syrup
6% Dry Solids and Concentrated 2X

| | Halogen Dry | | | | | Lactic | | Acetic | |
|---|---|---|---|---|---|---|---|---|---|
| Time | Solids | DP3 | DP2 | Dextrose | Xylose | acid | Glycerol | acid | Ethanol |
| hours | %, w/w | g/L | g/L | g/L | g/L | g/L | g/L | g/L | %, v/v |
| 0 | 14.5 | 0.70 | 3.44 | 43.60 | 5.75 | 0.32 | 1.70 | 0.74 | 0.13 |
| 6 | na | 0.84 | 2.32 | nd | 4.52 | 0.44 | 4.11 | 1.38 | 2.20 |

TABLE 11
Shake Flask Fermentation of C6 Syrup
9% Dry Solids, with Sequential Increase of 5% Dry Solids

| | Oven Dry | | | | | Lactic | | Acetic | |
|---|---|---|---|---|---|---|---|---|---|
| Time | Solids | DP3 | DP2 | Dextrose | Xylose | acid | Glycerol | acid | Ethanol |
| hours | %, w/w | g/L | g/L | g/L | g/L | g/L | g/L | g/L | % v/v |
| 0 | 12.7 | 0.94 | 5.84 | 62.00 | 8.13 | nd | 3.83 | 2.43 | 0.05 |
| 24 | na | 0.92 | 4.45 | 0.78 | 7.02 | 1.15 | 7.08 | 3.95 | 3.01 |

TABLE 12
Shake Flask Fermentation of C6 Syrup
9% Dry Solids and Concentrated 2X

| | Oven Dry | | | | | Lactic | | Acetic | |
|---|---|---|---|---|---|---|---|---|---|
| Time | Solids | DP3 | DP2 | Dextrose | Xylose | acid | Glycerol | acid | Ethanol |
| hours | %, w/w | g/L | g/L | g/L | g/L | g/L | g/L | g/L | %, v/v |
| 0 | 18.0 | 1.51 | 10.74 | 100.77 | 14.66 | 0.28 | 9.31 | 3.56 | 0.07 |
| 7 | na | 1.45 | 10.65 | 57.43 | nr | 0.46 | 8.11 | 3.51 | 2.07 |
| 24 | na | 1.74 | 8.60 | 1.36 | 15.05 | 0.75 | 13.37 | 5.09 | 6.00 |

TABLE 13
Shake Flask Fermentation of C6 Syrup
16.5% Dry Solids with Several Strains of Saccharomyces cerevisiae

| | | Oven Dry | Dex- | Xy- | Lac- | Glyc- | Ace- | Eth- |
|---|---|---|---|---|---|---|---|---|
| Time | | Solids | trose | lose | tic | erol | tic | anol |
| hours | Strain | (%) | g/L | g/L | g/L | g/L | g/L | %, v/v |
| 0 | None | 16.5 | 125.6 | 17.3 | 0.1 | 3.6 | 5.6 | 0.0 |
| 24 | 134-12 | Na | 1.6 | 12.7 | 0.6 | 8.8 | 5.2 | 6.8 |
| 24 | 424a | Na | 0.4 | 10.5 | 0.4 | 9.4 | 5.6 | 7.1 |
| 24 | ER2 | Na | 1.6 | 13.7 | 1.4 | 8.2 | 5.6 | 6.8 |
| 24 | Y500 | Na | 1.7 | 13.2 | 0.4 | 8.3 | 5.8 | 6.8 |
| 48 | 134-12 | Na | 1.4 | 8.4 | 0.7 | 8.8 | 6.2 | 6.9 |
| 48 | 424a | Na | 1.6 | 6 | 0.4 | 9.3 | 7.3 | 6.9 |
| 48 | ER2 | Na | 1.5 | 12.8 | 1.4 | 8.5 | 7.5 | 6.8 |
| 48 | Y500 | Na | 1.5 | 11.1 | 0.5 | 8.9 | 7.9 | 6.6 |

TABLE 14
Shake Flask Fermentation of C6 Syrup
20% Dry Solids with Recombinant Saccharomyces cerevisiae strain 424a

| | Halogen Dry | | | | | Lactic | | Acetic | |
|---|---|---|---|---|---|---|---|---|---|
| Time | Solids | DP3 | DP2 | Dextrose | Xylose | acid | Glycerol | acid | Ethanol |
| Hours | %, w/w | g/L | g/L | g/L | g/L | g/L | g/L | g/L | %, v/v |
| 29 | | 1.4 | 675 | 1.0 | 20.4 | nd | 17.1 | 0.9 | 8.3 |
| 48 | | 1.5 | 7.9 | 2.1 | 22.2 | nd | 16.5 | 0.9 | 8.0 |

An alternative process that can be used is referred to as simultaneous saccharification and fermentation (abbreviated: SSF). In such a process, the enzymatic digestion of the hemicellulose fraction 289 or the cellulose pulp fraction 218 is done in a medium that also includes the microorganisms. As the sugars are being released by the digestion process, they are consumed by the microorganisms for biomass accumulation and/or fermentation product production. Optionally a separate sugar source may also be fed to the digesting/fermentation mixture during the process. One benefit of an SSF process is that the consumption of the released sugars prevents feedback inhibition of any digesting enzymes that may be sensitive to feedback inhibition by the sugar. The SSF process can be carried out at a pH of 4-6 at 30-60° C. for 5 to 7 days depending on the enzyme dosing, composition of enzyme blend used, thermostability of the enzymes, thermal and inhibitor tolerance of the microorganisms used as well as the starting concentrations of dry solids in fermentation. A SSF shake flask experiment was done using the C6 syrup obtained from digesting the cellulose pulp 218 at high enzyme dose and high solids (20%) at 40° C. Results of SSF shake flask experiment are shown in Table 15, where the shake flasks with 20% w/w dry solids cellulose pulp 218 were not digested to the point of liquefaction in 24 hours and could not be sampled.

TABLE 15
Shake Flask Simultaneous Saccharification
and Fermentation at 40° C.

| | Dry | Dex- | Xy- | Lactic | Glyc- | Acetic | Eth- |
|---|---|---|---|---|---|---|---|
| Time | Solids | trose | lose | acid | erol | acid | anol |
| hours | %, w/w | g/L | g/L | g/L | g/L | g/L | %, v/v |
| 24 | 15 | 4.7 | 16.8 | 0.4 | 14.2 | 1.6 | 5.51 |
| 24 | 20 | Not liquefied | | | | | |
| 48 | 15 | 2.0 | 18.0 | 0.4 | 15.0 | 2.1 | 6.30 |
| 48 | 20 | 10.8 | 21.7 | 0.4 | 14.0 | 1.6 | 6.58 |
| 96 | 15 | 4.9 | 21.7 | 0.5 | 16.3 | 2.4 | 4.19 |
| 96 | 20 | 23.1 | 25.6 | 0.5 | 14.8 | 1.8 | 5.61 |
| 120 | 15 | 5.4 | 24.5 | 0.6 | 17.6 | 2.5 | 3.11 |
| 120 | 20 | 28.0 | 29.7 | 0.6 | 16.6 | 2.0 | 4.38 |

A variation of a SSF process, is a semi SSF process wherein the fermentation is conducted in stages, typically, but not necessarily with different feedstocks. In a first stage a typical SHF is conducted using as the feedstock a C5 or C6 syrup pre-prepared by hydrolysis of the soluble hemicellulose 289 and cellulose pulp 218. In this initial phase biomass is accumulated with or without making the desired fermentation product. In a second phase the fermentation media containing the accumulated biomass is added to medium containing the hemicellulose 289 or cellulose pulp 218 in the presence of the hydrolyzing enzymes so that fermentation of the released sugars is occurring simultaneously with their hydrolytic release by the enzymes.

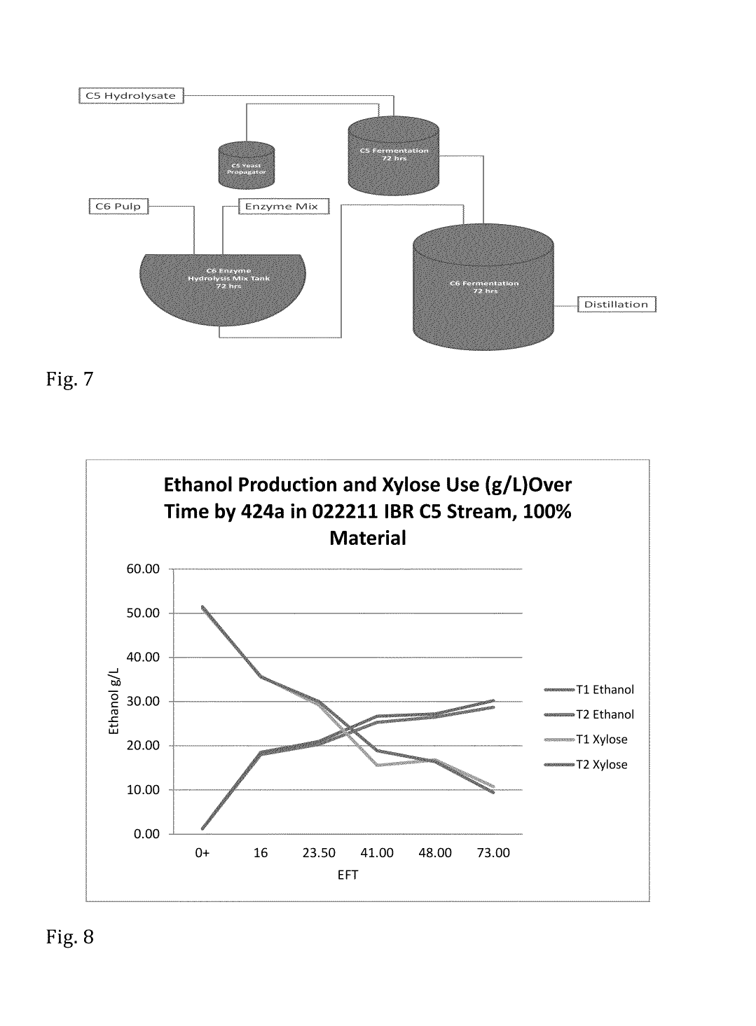

FIG. 7 illustrates one optimal method for a two stage semi-SSF process. In the first phase a first portion of C5 enriched syrup obtained from enzymatic hydrolysis of the soluble hemicellulose fraction 218, is used to accumulate biomass by aerobic growth in a microorganism propagator. In the illustrated embodiment, the yeast is a C5 competent ethanologen such as yeast strain 424a capable of producing ethanol from C5 sugars. The propagated yeast is then used to inoculate a fermentation media fed with a second portion of the C5 enriched syrup and grown anaerobically for a sufficient time to exhaust the sugars and produce a first portion of ethanol. FIG. 8 is a graph illustrating the time course for production of ethanol and simultaneous utilization of the C5 sugar xylose during an exemplary first stage conducted in laboratory shake flasks in duplicate.

Meanwhile, in preparation for the second phase, the cellulose pulp 218 made as described herein, is treated with a cellulolytic enzyme cocktail for a time sufficient to partly release a first portion of C6 sugars from the cellulose pulp 218. In the second phase, the yeast culture resulting from anaerobic fermentation on the C5 enriched syrup mentioned above is used to inoculate a larger medium containing the partly digested cellulose pulp and first portion of C6 sugars. This second phase of fermentation is continued under anaerobic conditions for a time sufficient to further hydrolyze the cellulose pulp into further C6 sugars and to produce ethanol. This method will produce a sufficient concentration of ethanol (at least 8% v/v) to make it economical for distillation and recovery.

Such a semi-SSF process was conducted in two stages in a laboratory test. The first stage used a fermentation broth obtained by fermentation of the xylose fermenting yeast 424a on a C5 syrup obtained from enzymatic digestion of a hemicellulose fraction 289 from corn stover in a non-baffled shake flask containing 50 ml of the detoxified C5 syrup. The C5 syrup was treated to remove toxic degradation products that are formed during the pretreatment such as furfural, hydroxymethyl furfural (HMF), phenolics, organic acids consisting primarily of acetic acid, and other organics by using a combination of solvent extraction to remove furfurlal, HMF and phenolics, ion-exchange chromatography using charged resins to remove acids, and/or evaporation to strip off volatile components. An inoculum of 25% was used for a second medium containing the C5 syrup in sealed flasks rotated at 100 rpms that was incubated at 30° C. under anaerobic growth conditions. After 72 h, the broth from this stage was used to inoculate 150 ml of a medium containing a corn stover cellulose pulp 218 that was pretreated for 72 hr with a cellulolytic enzyme cocktail. This cellulolytic cocktail consisted of enzymes described in paragraph 0055. As shown in Table 16, after 72 hr of fermentation of the C6 syrup/pulp, a production of about 8.8% v/v of ethanol was obtained in duplicate with a concomitant utilization of 98.5% of the available glucose and about 57% of the available xylose.

TABLE 16
Shake Flask Semi-Simultaneous Saccharification
and Fermentation of C5 Syrup and C6 Syrup

| | Glu- | Xy- | Lactic | Glyc- | Acetic | Eth- |
|---|---|---|---|---|---|---|
| | cose | lose | acid | erol | acid | anol |
| Time | g/L | g/L | g/L | g/L | g/L | %, v/v |
| 0 | 147.0 | 13.7 | 0.4 | 0.3 | 6.6 | |
| 72 | 2.0 | 7.3 | 2.5 | 7.3 | 8.0 | 8.4 |
| 72 | 2.4 | 8.1 | 2.0 | 8.1 | 8.7 | 8.8 |

The examples that follow are for purposes of illustration of steps taken in exemplary practices of certain aspects of the present disclosure and are not intended to limit or exemplify all ways in which the invention may be embodied by one of ordinary skill in the art.

### Example 1

### Acetic Acid/Ethyl Acetate Processing of Corn Stover

1.5 kg of corn chopped stover having 92% solids content (1380 grams) and 8% moisture was added to a jacketed rotary reactor. Fifteen-2.5 inch (500 g) ceramic balls and 7 liters of 70% acetic acid were added and the reactor was closed. Reactor rotation was started and steam injected into the jacket. In 10 minutes, the internal reactor temperature reached 165° C. The temperature was held for 2 minutes and then steam injection was discontinued. Steam was slowly released from the jacket to lower the internal temperature of the reactor to 150° C. over 3 minutes. The reactor was then allowed to cool over a period of ½ hour to 100° C. Thereafter, cooling water was added to bring the reactor temperature to 60° C. and the reactor was opened. The cooked stover was filtered over a Buchner funnel and pressed. Five liters of an acetic acid hydrolysate filtrate was collected. Five liters of 99% acetic acid warmed to a temperature of 50° C. was used to solubilize and to wash residual lignin and hemicellulose from the cake and collected separately. Four liters of ethyl acetate was added to wash the cake of acetic acid and the wash was filtered to obtain an ethyl acetate filtrate and cake. The cake was removed from the funnel, fluffed and air dried forming Sample A (810 grams).

The acetic acid first filtrate was evaporated to 1.2 liters. The second acetic acid filtrate was added to the first and evaporated again to a final volume of 1.2 liters. The ethyl acetate filtrate was added to the evaporated hydrolysate mixture and this was evaporated to a syrup of ˜800 ml. This warm syrup was added to 2 liters of ethyl acetate to precipitate out the hemicellulose and lignin (Sample B, 475 grams). The filtrate was concentrated to a heavy syrup and added to 600 ml ethyl acetate to precipitate another 50 grams of material (Sample C). The residual filtrate was evaporated to a heavy syrup containing 210 grams dissolved solids (Sample D). Ten grams of sample B was dispersed and put into 65 ml of hot water to dissolve the water soluble fraction then filtered and the filtrate was retained (Sample E).

These samples were analyzed for dissolved solids, hydrolyzed sugar forms, metals, N, P and K as well as acetic acid. The tables below summarizes the results for various analysis reported in g/Kg unless otherwise indicated.

TABLE 17
Dissolved solids for Sample A

| Sample ID A | Glucan | Xylan | Mannan | Galactan | ASH |
|---|---|---|---|---|---|
| pulp AS IS | 530.3 | 106.8 | 6.9 | 22.7 | 96.4 |
| pulp Dry Basis | 532.4 | 107.2 | 6.9 | 22.7 | 96.7 |

| Acid | Acid | | | | | |
|---|---|---|---|---|---|---|
| Insoluble | Soluble | Free | Free | Bound | Free | Dry |
| Lignin | Lignin | Dextrose | Xylose | Acetate | Acetate* | Solids* |
| 39.8 | 12.1 | 0.3 | 0.7 | 29.7 | 15.4 | 996.2 |
| 39.9 | 12.2 | 0.3 | 0.7 | 29.8 | | |

TABLE 18
Inorganic elements for Samples B-D

| Sample | Al | P | S | Zn | Co | Ni | Fe | Cr | Mg |
|---|---|---|---|---|---|---|---|---|---|
| Name | mg/kg | mg/kg | mg/kg | mg/kg | mg/kg | mg/kg | mg/kg | mg/kg | mg/kg |
| B | 123 | 4220 | 1970 | 69.1 | 1.25 | 3.74 | 1330 | 28.5 | 6700 |
| C | 14.8 | 556 | 1040 | 35.0 | 0.358 | 1.28 | 171 | 8.87 | 1120 |
| D | 0.289 | 94.0 | 297 | 4.12 | nd | nd | 2.58 | 0.604 | 38.0 |

| Ca | Cu | Na | K | Mn | Mo | B | N | Ash |
|---|---|---|---|---|---|---|---|---|
| mg/kg | mg/kg | mg/kg | mg/kg | mg/kg | mg/kg | mg/kg | % | % |
| 5720 | 13.6 | 43.9 | 44800 | 159 | 0.907 | 16.8 | 1.63 | 8.9% |
| 1590 | 25.5 | 52.2 | 43500 | 24.8 | 0.586 | 6.27 | 1.65 | 9.1% |
| 147 | 4.96 | 29.1 | 18600 | 1.12 | nd | 0.604 | 0.411 | 3.6% |

TABLE 19
Sugar analysis Samples B-D

| | C5 sugars | C6 sugars | C5 sugars | C6 sugars | Acid |
|---|---|---|---|---|---|
| | (as is) | (as is) | (hydrolyzed) | (hydrolyzed) | insolubles* |
| Sample info | mg/kg | mg/kg | mg/kg | mg/kg | % |
| Sample B: | 42,215 | 36,109 | 354,960 | 99,387 | 25.8 |
| lignin/hemicellulose | | | | | (2.61% N; |
| precipitate 1 | | | | | 4.6% Ash) |
| Sample C: | 57,034 | 34,067 | 249,980 | 72,546 | 45.5 |
| lignin/hemicellulose | | | | | (1.59% N; |
| precipitate 2 | | | | | 2.2% Ash) |
| Sample D: | 18,355 | 11,450 | 41,235 | 11,457 | 32.7** |
| residual syrup | | | | | (0.59% N; |
| (49.75% DS) | | | | | 6.3% Ash) |

TABLE 20
Miscellaneous analysis Samples B-D

| | | | Other | | Acetic | Ethyl | | |
|---|---|---|---|---|---|---|---|---|
| Ash | Sulfur | Potassium | metals | Nitrogen | Acid | Acetate | HMF + AcMF | Furfural* |
| % | g/kg | g/kg | g/kg | % | g/kg | g/kg | g/kg | g/kg |
| 8.91 | 1.97 | 44.80 | 18.43 | 1.63 | 76.8 | NA | 1.68 | 0.34 |
| (1.14 acid | | | | | | | | |
| insoluble) | | | | | | | | |
| 9.14 | 1.04 | 43.50 | 3.61 | 1.65 | 55.8 | NA | 9.43 | 0.29 |
| (1.00 acid | | | | | | | | |
| insoluble) | | | | | | | | |
| 3.63 | 0.30 | 18.60 | 0.32 | 0.41 | 301.7 | 181.5 | 20.18 | 6.98 |
| (2.06 acid | | | | | | | | |
| insoluble) | | | | | | | | |

*HMF = HydroxyMethylFurfural, AcMF = AcetoxyMethylFurfural (acetic ester of HMF)

TABLE 21
Sugars, lignin, acetic acid and elements in Sample E

| | | C5 | C6 | C5 | C6 | | | | | | | |
|---|---|---|---|---|---|---|---|---|---|---|---|---|
| | | sugars | sugars | sugars | sugars | | | | Other | | Acetic | Soluble |
| Sample | D.S. | (as is) | (as is) | (hydrolyzed) | (hydrolyzed) | Ash | S | K | metals | N | Acid | Lignin |
| info* | % | mg/kg | mg/kg | mg/kg | mg/kg | % | g/kg | g/kg | g/kg | % | g/kg | g/kg |
| Sample E - | 11.4 | 6,919 | 5,763 | 43,360 | 12,204 | 1.2 | 0.2 | 5.7 | 7.9 | 0.2 | 12.2 | 2.8 |
| aqueous | | | | | | | | | | | | |
| fraction | | | | | | | | | | | | |

### Example 2

### Acetic Acid/Ethyl Acetate Processing of Wheat Straw

1.7 kg of chopped wheat straw was combined with 9 liters 70% acetic acid (v/v in water) and heated in a tumbling steam-jacketed reactor for 5 minutes between 160 and 170° C. Twenty one ceramic balls (approximately 2 inch diameter) were added to aid mixing. The reactor was allowed to cool to a temperature between 60 and 90° C. under ambient conditions. The reaction product was filtered while still warm and washed with 70% acetic acid. The acetylated cellulose pulp (950 g dry weight) was collected accounting for 56% of the initial straw weight.

The liquid acetic acid hydrolysate was concentrated by evaporation to 40-50% dry solids and added to approximately four volumes chilled ethyl acetate. The precipitate containing lignin and hemicellulose was collected by filtration, washed with ethyl acetate, and air-dried. The dry precipitate was added to 90-92° C. water (approximately 1:4 w/w) with vigorous stirring and allowed to cool under ambient conditions to room temperature with continued stirring. The solid lignin was collected by filtration and washed with water. The solubilized hemicellulose was contained in the aqueous filtrate fraction.

### Example 3

### Acetic Acid/Ethyl Acetate Processing of Bamboo

2.0 kg chopped bamboo was combined with 10 kg 70% acetic acid (w/w in water) and heated in a tumbling steam jacketed reactor for 10 minutes to between 160 and 170 C. Again, 21 ceramic balls (approximately 2 inch diameter) were added to aid mixing. The reactor was allowed to cool under ambient conditions for 30 minutes until the temperature reached 120 C then rapidly cooled to 80-90 C. The reaction product was filtered while still warm and washed with 75% acetic acid (w/w in water) and water. The retained acetylated cellulose pulp was subsequently washed with ethyl acetate and dried and accounted for 52% of the initial bamboo weight.

### SELECTED REFERENCES

K. P. Vogel, J. F. Pederson, S. D. Masterson, J. J. Troy. Evaluation of a filter bag system for NDF, ADF and IVDMD forage analysis. Crop Scie. 39, 276-279 (1999).
J. B. Sluiter, R. O. Ruiz, C. J. Scarlata, A. D. Sluiter, D. W. Templeton. Compositional analysis of lignocellulosic feedstocks. 1. Review and description of methods. J. Agric. Food Chem. 58, 9043-9053 (2010).
M. Sedlak, H. J. Edenberg, N. W. Y Ho. DNA microarray analysis of the expression of the genes encoding the major enzymes in ethanol production during glucose and xylose co-fermentation by metabolically engineered Saccharomyces host. Enz. Microbial Technol. 33, 19-28 (2003).
S. C. Bate, W. A. Rogerson, F. G. Peach 1953. Improvements in the production of cellulose and cellulose derivatives. Great Britain Patent 686311.
P. P. Rousu, J. R. Antilla, E. J. Rousu 2001. Method for the recovery of formic acid. U.S. Pat. No. 6,252,109.
Alalouf O, Balazs Y, Volkinshtein M, Grimpel Y, Shoham G, Shoham Y. A new family of carbohydrate esterases is represented by a GDSL hydrolase/acetylxylan esterase from Geobacillus stearothermophilus. J Biol Chem. 2011 286(49):41993-2001.
Peter Biely. Microbial Carbohydrate Esterases Deacetylating Plant polysaccharides, Biotechnology Advances, 2012 online publication.

What is claimed is:

1. A method of fermentation to make a desired fermentation product comprising,
a. obtaining at least one of an acylated cellulose pulp and acylated hemicellulose fraction;
b. deacylating the acylated cellulose pulp and/or the acylated hemicellulose fraction;
c. contacting the deacylated acylated cellulose pulp and/or acylated hemicellulose fraction with an enzyme cocktail comprising a mixture of at least two enzymes selected from the group consisting of a cellulase and a hemicellulase enzyme for a time sufficient to form a syrup comprised predominantly of C5 or C6 sugars; and
d. growing a microorganism on the sugars to produce the desired fermentation product.

2. The method of claim 1 wherein deacylating the acylated cellulose pulp comprises contacting the obtained acylated cellulose pulp with a base.

3. The method of claim 1 wherein deacylating the acylated cellulose pulp comprises contacting the obtained acylated cellulose pulp with an esterase.

4. The method of claim 3 wherein contacting the acylated cellulose pulp with an esterase enzyme includes simultaneously contacting with the enzyme cocktail containing at least a hemicellulase and a cellulase enzyme according to step c and wherein the esterase is supplemental to endogenous esterase contained in the enzyme cocktail.

5. The method of claim 1 wherein contacting the acylated cellulose pulp or acylated hemicellulose in the presence of the esterase, hemicellulase or cellulase enzyme includes contacting in the presence of between 0.05% and 5% v/wt of a non-ionic detergent measured as a percentage of total weight of material present.

6. The method of claim 1 wherein the acylated cellulose pulp or acylated hemicellulose is predominantly acetylated cellulose or acetylated hemicellulose.

7. The method of claim 1 wherein growing the microorganism is done under conditions optimal to propagate the microorganism.

8. The method of claim 1 wherein growing the microorganism is done under conditions optimal to produce the desired product by the microorganism.

9. The method of claim 1 wherein the desired product is ethanol and the microorganism is selected from the group consisting of Zymomonas mobilis and a yeast.

10. The method of claim 9 where the microorganism is a yeast and the growing is done under aerobic conditions selected to propagate the yeast.

11. The method of claim 9 where the microorganism is a yeast genetically engineered to ferment C5 sugars to form ethanol, and the growing is done under anaerobic conditions selected to produce ethanol.

12. The method of claim 1 wherein the desired product is selected from the group consisting of an amino acid and an organic acid.

13. The method of claim 12 wherein the amino acid is selected from the group consisting of lysine and threonine and the microorganism is selected from the group consisting of Escherichia coli and Corynebacterium glutanicum.

14. The method of claim 12 wherein the desired product is an organic acid selected from the group consisting of lactic acid, gluconic acid, citric acid, malic acid, fumaric acid and succinic acid, and the microorganism is a fungus selected from the group consisting of Rhizopus, Schizosaccharomyces, Mucor, and Aspergillus.

15. The method of claim 1 wherein obtaining the acylated cellulose pulp and/or acylated hemicellulose fraction is done according to the method of processing lignocellulosic biomass to form an acylated cellulose pulp, comprising:
a. contacting a lignocellulosic biomass with a first amount of a C₁-C₂ acid selected from the group consisting of acetic acid, formic acid and mixtures of the same;
b. heating the contacted lignocellulosic biomass to a temperature and for a time sufficient to hydrolytically release a first portion of hemicellulose and lignin, forming a hydrolysate liquid and an acylated lignocellulose cake;
c. separating the acylated lignocellulosic cake from the first hydrolysate liquid;
d. contacting the acylated lignocellulose cake with a second amount of the C₁-C₂ acid to wash hemicellulose and lignin from the acylated lignocellulosic cake and separating the acid wash liquid from the acid washed acylated lignocellulosic cake; and
e. contacting the acid washed acylated lignocellulose cake with a first amount of a C₁-C₂ acid-miscible organic solvent to wash the C₁-C₂ acid, hemicellulose and lignin from the acid washed acylated lignocellulosic cake resulting in an acylated cellulose pulp and separating the C₁-C₂ acid-miscible solvent wash liquid from the solvent washed acylated cellulose pulp.

16. The method of claim 1 wherein obtaining the acylated cellulose pulp and/or acylated hemicellulose fraction is done according to the method of processing lignocellulosic biomass to form an acylated cellulose pulp, comprising:
a. contacting a lignocellulosic biomass with a first amount of a C₁-C₂ acid selected from the group consisting of acetic acid, formic acid and mixtures of the same;
b. heating the contacted lignocellulosic biomass to a temperature and for a time sufficient to hydrolytically release a first portion of hemicellulose and lignin, forming a hydrolysate liquid and an acylated lignocellulose cake;
c. separating the acylated lignocellulosic cake from the first hydrolysate liquid;
d. contacting the acylated lignocellulose cake with a second amount of the C₁-C₂ acid to wash hemicellulose and lignin from the acylated lignocellulosic cake and separating the acid wash liquid from the acid washed acylated lignocellulosic cake;
e. contacting the acid washed acylated lignocellulose cake with a first amount of a C₁-C₂ acid-miscible organic solvent to wash the C₁-C₂ acid, hemicellulose and lignin from the acid washed acylated lignocellulosic cake resulting in an acylated cellulose pulp and separating the C₁-C₂ acid-miscible solvent wash liquid from the solvent washed acylated cellulose pulp;
f. combining the solvent wash liquid with at least one of the hydrolysate and the acid wash liquid forming an acidic organic solvent extract;
g. condensing the acidic organic solvent extract forming an acidic organic solvent syrup enriched with hemicellulose and lignin;
h. adding a second amount of the C₁-C₂ acid-miscible organic solvent to the acidic organic solvent syrup, the second amount being sufficient to form a precipitate comprised of hemicellulose and lignin;
i. separating the hemicellulose and lignin precipitate from the acidic organic solvent syrup;
j. dissolving the precipitate with an aqueous solvent to form a solution of solubilized hemicellulose and insoluble lignin; and
k. separating the insoluble lignin from the solubilized hemicellulose.

17. The method of claim 15 wherein deacylating the acylated cellulose pulp comprises contacting the obtained acylated cellulose pulp with a base.

18. The method of claim 16 wherein deacylating the acylated cellulose pulp comprises contacting the obtained acylated cellulose pulp with a base.

19. The method of claim 15 wherein deacylating the acylated cellulose pulp comprises contacting the obtained acylated cellulose pulp with an esterase.

20. The method of claim 16 wherein deacylating the acylated cellulose pulp comprises contacting the obtained acylated cellulose pulp with an esterase.
